(19) United States
(12) Patent Application Publication
(10) Pub. No.: US 2024/0399048 A1
(43) Pub. Date: Dec. 5, 2024

(54) METHOD AND APPARATUS FOR A SUCTION CONNECTION SYSTEM

(71) Applicant: Dignity Health, San Francisco, CA (US)

(72) Inventors: Dakota Graham, San Francisco, CA (US); Arnau Benet, San Francisco, CA (US)

(21) Appl. No.: 18/695,284

(22) PCT Filed: Oct. 26, 2022
(86) PCT No.: PCT/US2022/078737

§ 371 Date: Mar. 25, 2024

Related U.S. Application Data
(60) Provisional application No. 63/272,053, filed on Oct. 26, 2021.

(51) Int. Cl. A61M 1/00; A61M 25/01

(52) U.S. Cl. CPC A61M 1/86; A61M 25/0111

(57) ABSTRACT

A catheter placement system for drawing a medical tube through a sheath includes a suction generation device and further includes one or more suction adapter devices that enable connection of the suction generation device to the sheath and facilitate insertion of the medical tube into the sheath. The one or more suction adapter devices are operable for lateral removal from the sheath and the medical tube, enabling connection of the medical tube to one or more external devices prior to drawing the medical tube through the sheath.

### CROSS-REFERENCE TO RELATED APPLICATIONS

This is a PCT patent application that claims benefit to U.S. Provisional Patent Application Ser. No. 63/272,053 filed 26 Oct. 2021, which is herein incorporated by reference in its entirety.

### FIELD

The present disclosure generally relates to a medical device, and in particular to method and apparatus for a suction connection system.

### BACKGROUND

In many neurosurgical procedures, catheters are used to allow excess fluids built-up in the brain to drain in another portion of the body, normally into the abdomen during a ventriculoperitoneal shunt placement. During a ventriculoperitoneal shunt placement, the distal catheter, which connects the shunt valve to the target cavity, is passed from a cranial incision to an abdominal incision subcutaneously. Catheters are fed through a plastic tube, or sheath, that has been previously inserted using a tunneler stylet. The catheter is then manually fed through the plastic tube. This process is time consuming, burdensome, and may require several attempts.

It is with these observations in mind, among others, that various aspects of the present disclosure were conceived and developed.

### BRIEF DESCRIPTION OF THE DRAWINGS

FIG. 1A is an illustration showing placement of a medical tube within a body aided by a catheter placement system;

FIG. 1B is an illustration showing placement of a medical tube within a body aided by the catheter placement system of FIG. 1A following removal of a suction generation device and suction adapter devices with a sheath and a medical tube positioned within the body;

FIG. 1C is an illustration showing the medical tube positioned within the body following removal of the sheath of the catheter placement system of FIG. 1A;

FIG. 2A is a perspective view showing an embodiment of the catheter placement system of FIG. 1A including a single suction adapter device;

FIG. 2B is an enlarged view of the catheter placement system of FIG. 2A showing engagement of the suction adapter device with a sheath and a suction tube;

FIG. 2C is a perspective view showing an embodiment of the catheter placement system of FIG. 1A including two suction adapter devices;

FIG. 2D is an exploded view showing the catheter placement system of FIG. 2C;

FIG. 2E is an enlarged view showing engagement of the two suction adapter devices with a sheath and a suction tube of the catheter placement system of FIG. 2C and accepting a medical tube;

FIG. 2F is an enlarged view showing the medical tube of FIG. 2E positioned within the suction adapter devices after being drawn through the sheath;

FIG. 2G is a perspective view showing decoupling of various components of the catheter placement system of FIG. 2C;

FIG. 3A is a front perspective view showing a first embodiment of a suction adapter device according to the catheter placement system of FIGS. 1A-1C;

FIG. 3B is a rear perspective view showing the suction adapter device of FIG. 3A;

FIG. 3C is a plan view showing a first section and a second section of the suction adapter device of FIG. 3A;

FIG. 4A is a front perspective view showing a second embodiment of a suction adapter device according to the catheter placement system of FIGS. 1A-1C;

FIG. 4B is a rear perspective view showing the suction adapter device of FIG. 4A;

FIG. 4C is a plan view showing a first section and a second section of the suction adapter device of FIG. 4A;

FIG. 5A is a front perspective view showing a third embodiment of a suction adapter device according to the catheter placement system of FIGS. 1A-1C in a closed configuration;

FIG. 5B is a front perspective view showing the suction adapter device of FIG. 5A in an open configuration;

FIG. 5C is a plan view showing a first section and a second section of the suction adapter device of FIG. 5A;

FIG. 6A is a front perspective view showing a fourth embodiment of a suction adapter device according to the catheter placement system of FIGS. 1A-1C in a closed configuration;

FIG. 6B is a front perspective view showing the suction adapter device of FIG. 6A in an open configuration;

FIG. 6C is a plan view showing a first section and a second section of the suction adapter device of FIG. 6A;

FIG. 7A is a front perspective view showing a fifth embodiment of a suction adapter device according to the catheter placement system of FIGS. 1A-1C in a closed configuration;

FIG. 7B is a front perspective view showing the suction adapter device of FIG. 7A in an open configuration;

FIG. 7C is a plan view showing a first section and a second section of the suction adapter device of FIG. 7A;

FIG. 8A is a front perspective view showing a sixth embodiment of a suction adapter device according to the catheter placement system of FIGS. 1A-1C in a closed configuration;

FIG. 8B is a plan view showing a first section and a second section of the suction adapter device of FIG. 8A;

FIG. 8C is an enlarged view showing engagement of the suction adapter device of FIG. 8A with a sheath and a suction tube of the catheter placement system of FIG. 2C and accepting a medical tube;

FIG. 9A is a first process flow diagram showing a method for drawing the medical tube through the sheath by the catheter placement system and suction adapter devices of FIGS. 1A-8C;

FIG. 9B is a second process flow diagram continuing the method of FIG. 9A for drawing the medical tube through the sheath by the catheter placement system and suction adapter devices of FIGS. 1A-8C; and

FIG. 9C is a third process flow diagram continuing the method of FIGS. 9A and 9B for drawing the medical tube through the sheath by the catheter placement system and suction adapter devices of FIGS. 1A-8C.

Corresponding reference characters indicate corresponding elements among the view of the drawings. The headings used in the figures do not limit the scope of the claims.

### DETAILED DESCRIPTION

Various embodiments of a catheter placement system including a suction adapter device are described herein. In particular, the suction adapter device of the catheter placement system enables connection of a suction device to aid practitioners in passing a medical tube (e.g., a catheter) from a first incision to a second incision within a body.

### INTRODUCTION

To place a medical tube within a body in conventional practice, for example during a ventriculoperitoneal shunt placement, a practitioner makes a first incision near a head of the body and a second incision near an abdomen of the body. The practitioner then creates a “tunnel” within the body between the first incision and the second incision, usually with a rigid “tunneler” device positioned within a sheath. In particular, the practitioner inserts the sheath with the rigid tunneler device into the first incision and through the body until the sheath emerges from the body at the second incision. Following placement of the sheath within the body, the rigid tunneler device can be removed with the sheath still positioned within the body.

The following steps of a conventional medical tube placement procedure tend to frustrate practitioners and can take considerable amounts of time. To place the medical tube within the body, practitioners insert a first end of the medical tube into the sheath at a first portion of the sheath and gently “push” or otherwise guide the medical tube through the sheath until the first end of the medical tube emerges at a second portion of the sheath. The sheath can be between 2 and 3 feet long, depending on the dimensions of the body, and the medical tube is longer than the sheath. Practitioners often find themselves resorting to sub-optimal methods to guide the medical tube through the sheath, such as rolling up the medical tube like a “lasso” and using a combination of fingers and saline solution to guide the medical tube through the sheath. This step can take multiple tries and poses considerable risks due to the time consumed (around 20 minutes) and potential damage to the medical tube. Following successful guidance of the medical tube through the sheath, the sheath can be removed from the body with the medical tube remaining within the body.

### Catheter Placement System

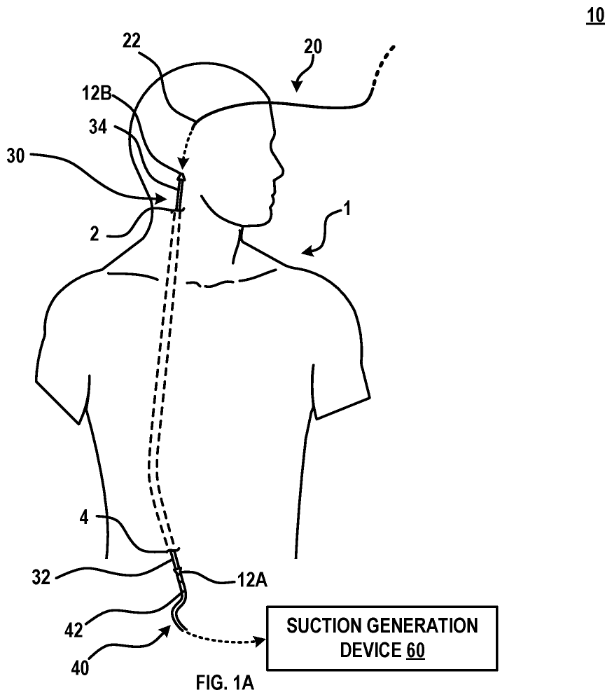

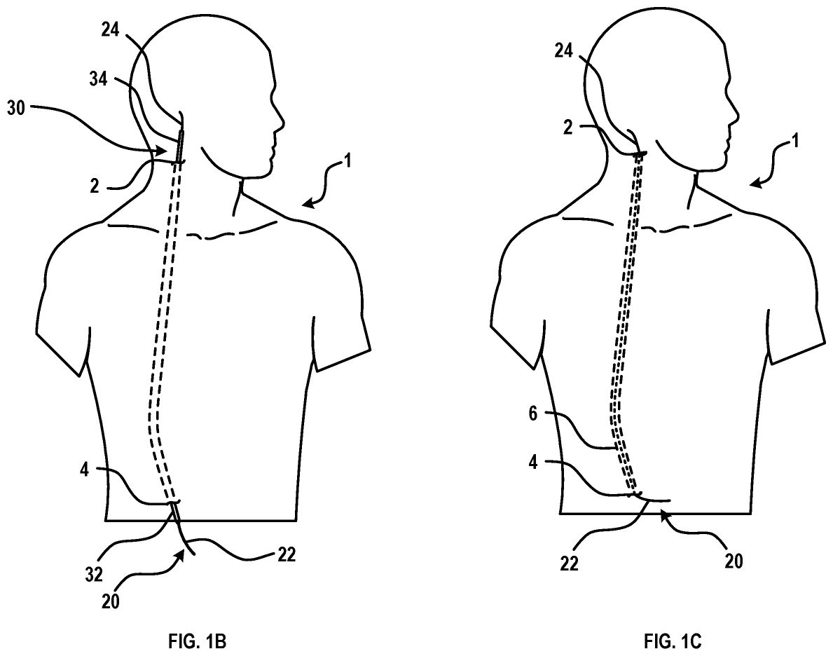

As such, the catheter placement system including the suction adapter device described herein is directed to solving the problem of guiding a medical tube through a sheath for placement within the body. The catheter placement system includes the suction adapter device that enables connection of the sheath of the catheter placement system with a suction generation device that draws the medical tube from a first portion of the sheath to a second portion of the sheath. FIGS. 1A-1C show the catheter placement system (hereinafter, system 10) for placement of a medical tube 20 within a body 1. The system 10 includes a sheath 30 configured for placement within the body 1 as described above, and further includes a first suction adapter device (shown in FIG. 1A as a first suction adapter device 12A, but can be one of several embodiments of the suction adapter device 100, 200, 300, 400 or 500 described in greater detail herein) that connects the sheath 30 to a suction tube 40 in fluid flow communication with a suction generation device 60. As shown, the sheath 30 includes a first sheath portion 32 and a second sheath portion 34 and is configured for receipt of the medical tube 20 for placement within the body 1. The suction generation device 60 is configured to create a suction force that draws air and the medical tube 20 from the first sheath portion 32 of the sheath 30 to the second sheath portion 34 of the sheath 30. The suction generation device 60 and the suction tube 40 can be part of a conventional suction generation device used in operating rooms; note that in practice, the suction tube 40 can have a greater diameter than the sheath 30.

With specific reference to FIG. 1A, the sheath 30 can first be positioned within the body 1 by insertion of a first sheath portion 32 of the sheath 30 into a first incision 2 of the body 1 and by tunneling of the sheath 30 through the body 1 to a second incision 4 of the body 1 as described above such that the first sheath portion 32 of the sheath 30 is exposed at the second incision 4 and a second sheath portion 34 of the sheath 30 is exposed at the first incision 2. Following insertion of the sheath 30 within the body 1, a practitioner can couple the first suction adapter device 12A with the first sheath portion 32 of the sheath 30 as shown. The practitioner can then couple the first suction adapter device 12A with a first portion 42 of the suction tube 40 in fluid flow communication with the suction generation device 60. The practitioner can then insert a first portion 22 of the medical tube 20 into the second sheath portion 34 of the sheath 30 and activate the suction generation device 60 such that the first portion 22 of the medical tube 20 is consequently drawn from the second sheath portion 34 of the sheath 30 and through the body 1 until the first portion 22 of the medical tube 20 meets the first sheath portion 32 of the sheath 30 at the second incision 4 and a second portion 24 of the medical tube 20 extends from the second sheath portion 34 of the sheath 30 at the first incision 2.

In some embodiments, the system 10 can include a second suction adapter device (shown in FIG. 1A as a second suction adapter device 12B, but can be one of several embodiments of the suction adapter device 100, 200, 300, 400 or 500 described in greater detail herein) which can couple with the second sheath portion 34 of the sheath 30 following positioning of the sheath 30 within the body 1 to improve suction (e.g., to maximize the suction force generated through the sheath 30 by the suction generation device 60). In one aspect, the inclusion of the first suction adapter device 12A and/or the second suction adapter device 12B within the system 10 effectively reduces a “diameter” of the sheath 30 around the medical tube 20 and adapts the suction tube 40 to the sheath 30 to maximize the suction force within the sheath 30 generated at the suction generation device 60. Without inclusion of the first suction adapter device 12A and/or the second suction adapter device 12B, the suction force present within the sheath 30 would not be sufficient enough to draw the medical tube 20 through the sheath 30.

With reference to FIGS. 1B and 1C, following successful insertion of the medical tube 20 through the sheath 30 as discussed above, the first suction adapter device 12A, the second suction adapter device 12B, the suction tube 40 and the suction generation device 60 can be removed from the sheath 30. The sheath 30 can then be removed from the body 1, leaving the medical tube 20 within the body 1 as shown in FIG. 1C.

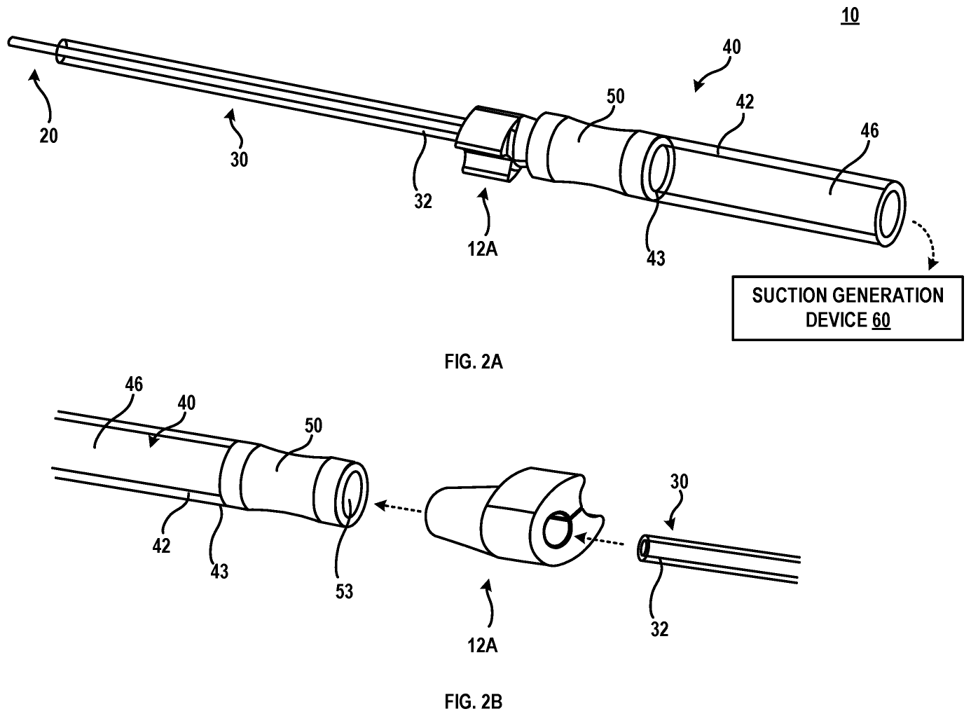

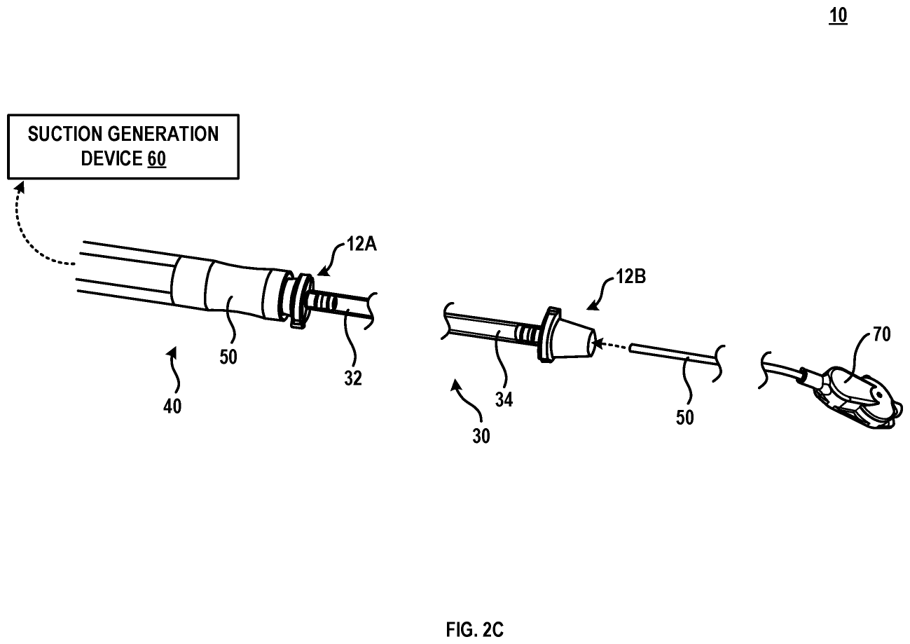

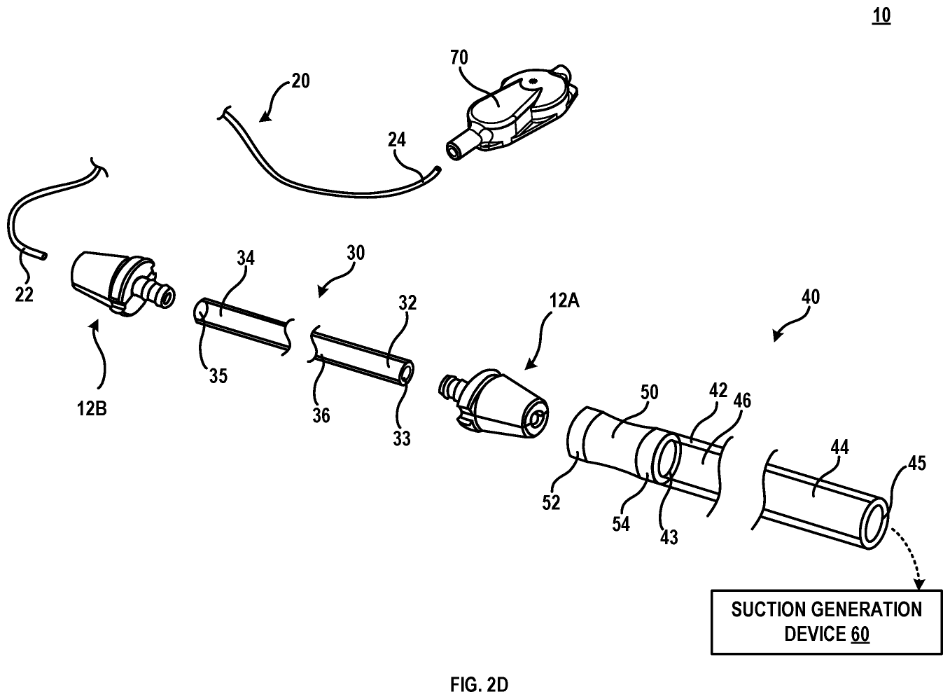

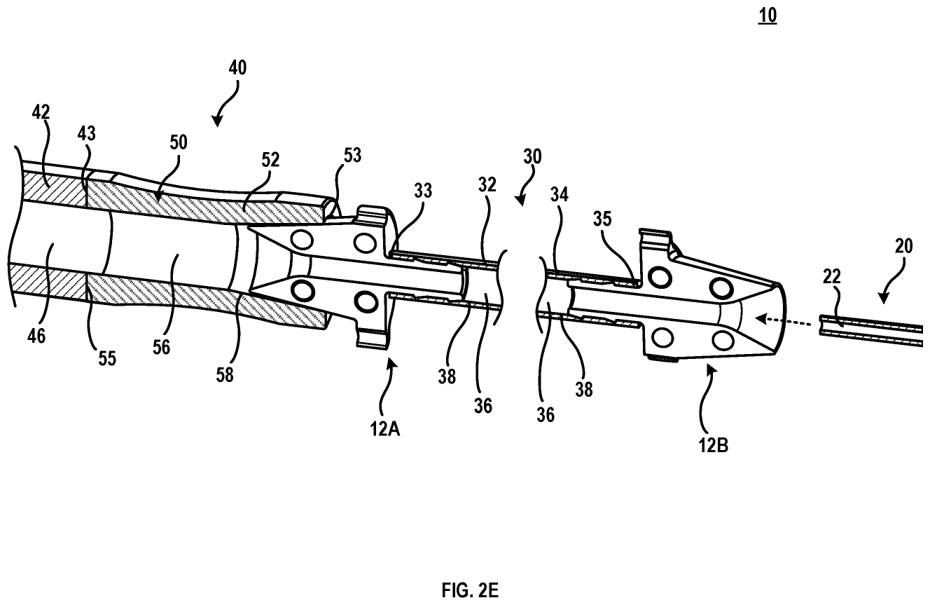

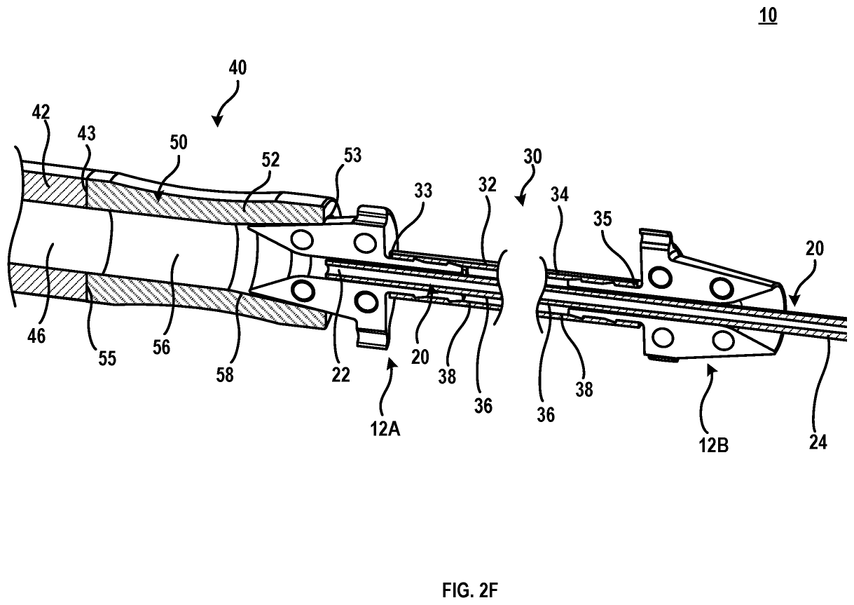

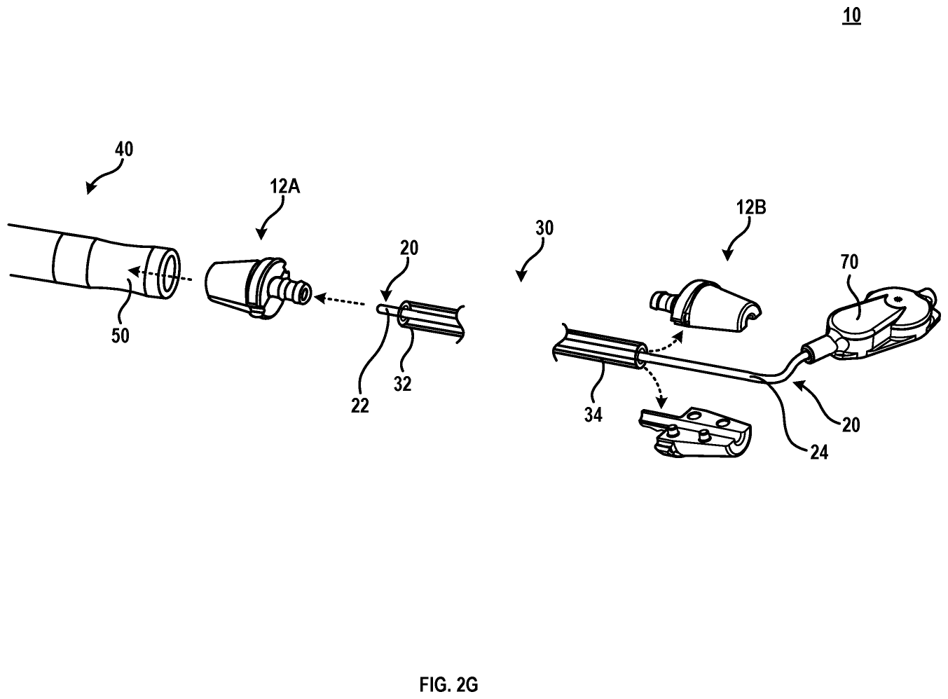

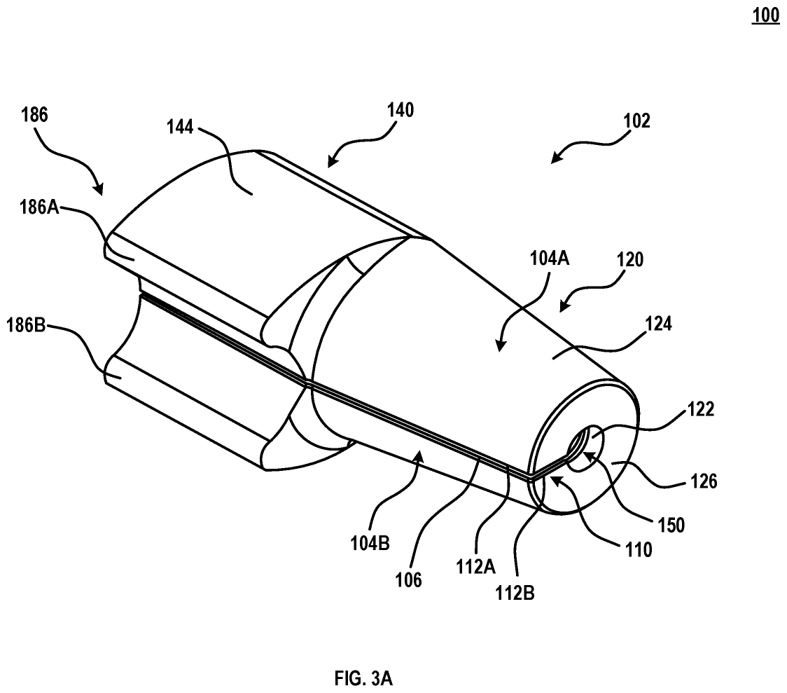

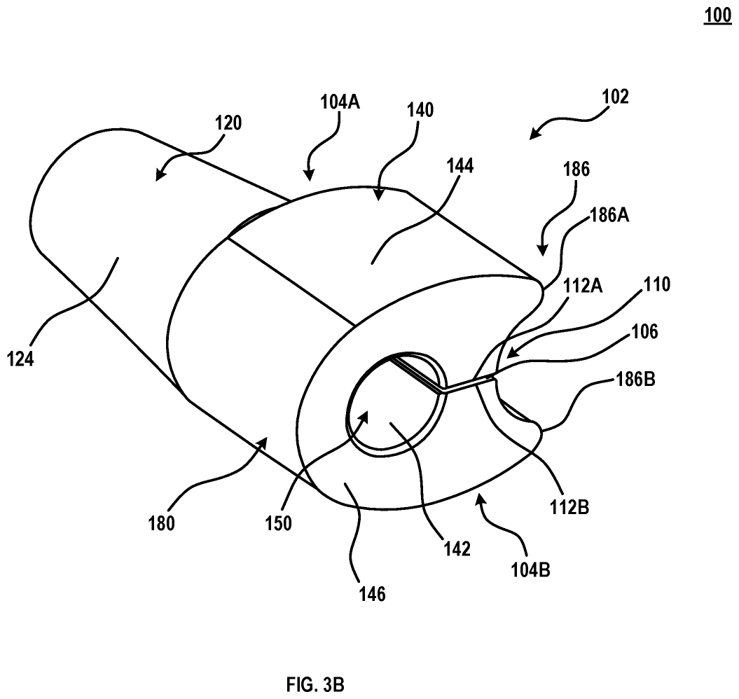

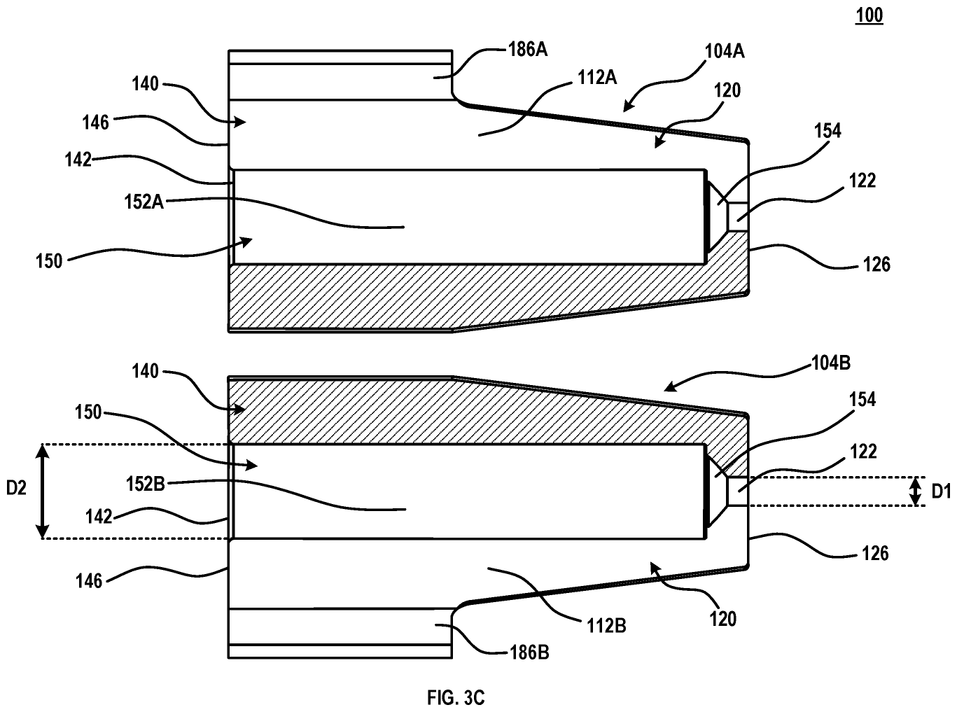

FIGS. 2A-2G show various embodiments of the system 10; in particular, FIGS. 2A and 2B show a first example implementation of the system 10 featuring the first suction adapter device 12A (in the example shown, the first suction adapter device corresponds to a first embodiment of a suction adapter device 100 discussed in greater detail herein with respect to FIGS. 3A-3C). As discussed above, the first suction adapter device 12A can be positioned between the sheath 30 and the suction tube 40 to create an airtight seal between the sheath 30 and the suction tube 40. In some embodiments, the suction tube 40 can include a suction tube connector 50 positioned at the first portion 42 of the suction tube 40 that receives the first suction adapter device 12A as shown. The suction tube connector 50 can include a first connector aperture 53 that communicates with the first portion 42 of the suction tube 40. In particular, the suction tube 40 includes a first suction tube aperture 43 that communicates with a suction tube lumen 46, and the first connector aperture 53 of the suction tube connector 50 communicates with the suction tube lumen 46.

As shown, the first suction adapter device 12A can be inserted directly into the first connector aperture 53 of the suction tube connector 50. The first suction adapter device 12A can also couple with the first sheath portion 32 of the sheath 30 to connect the sheath 30 with the suction tube 40. In one implementation, the first suction adapter device 12A couples with the first sheath portion 32 of the sheath 30 by insertion of the first sheath portion 32 of the sheath 30 into the first suction adapter device 12A, however other embodiments are also discussed herein that involve a different coupling arrangement.

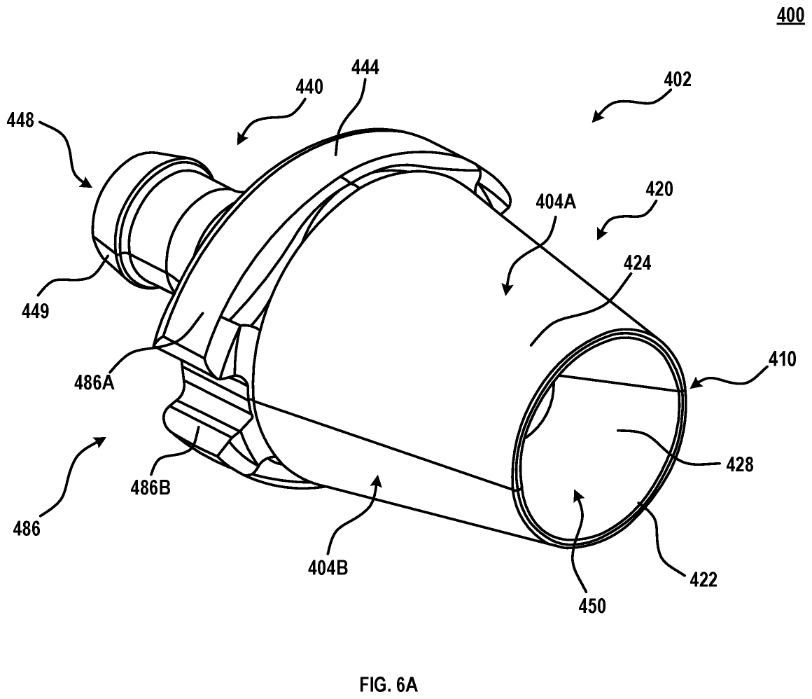

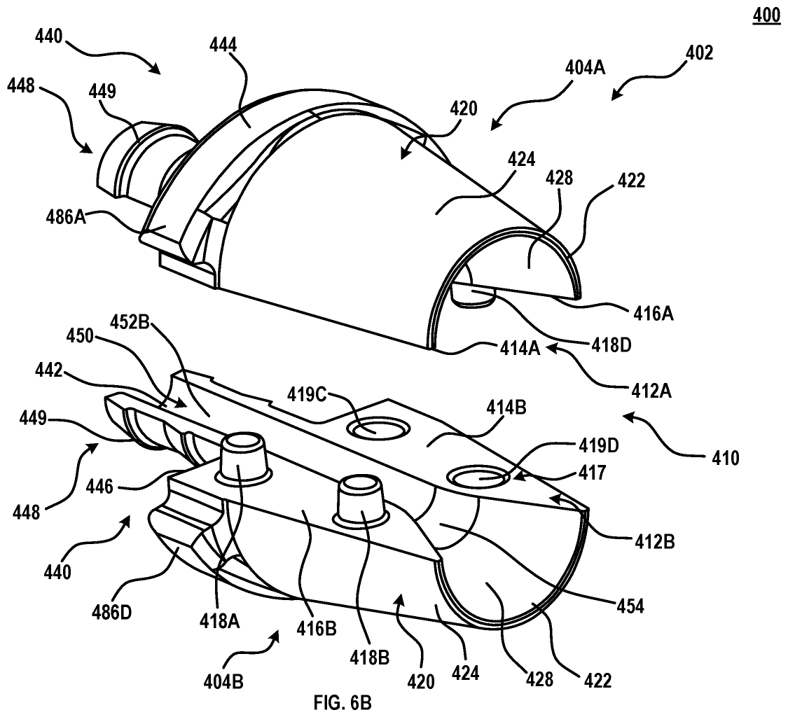

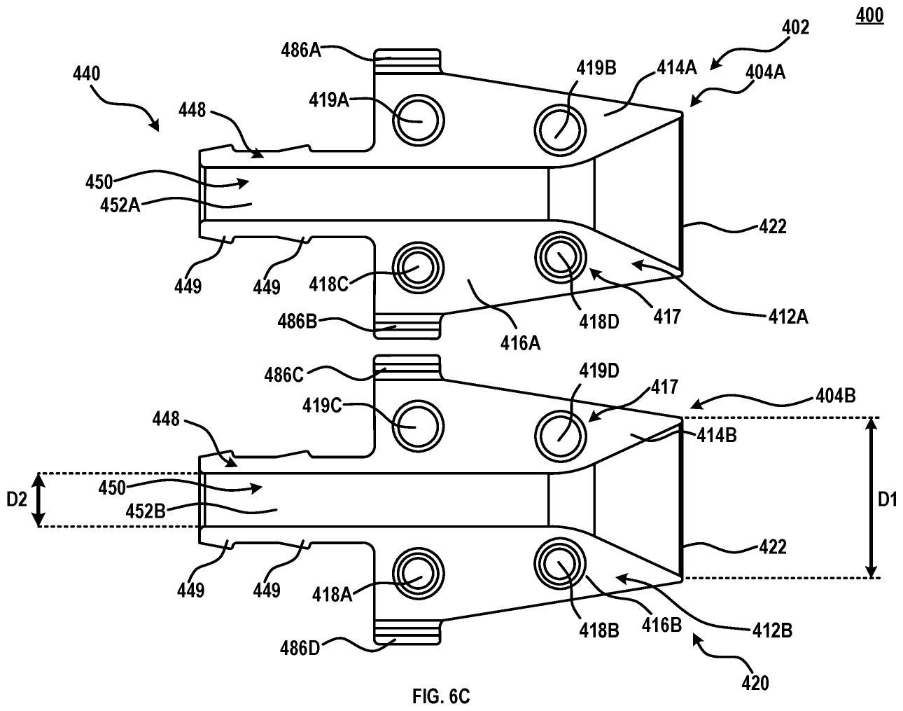

FIGS. 2C-2G show another example implementation of the system 10 including the first suction adapter device 12A that couples the suction tube 40 (at the suction tube connector 50) and the first sheath portion 32 of the sheath 30. The system 10 can also include the second suction adapter device 12B that couples with the second sheath portion 34 of the sheath 30 as shown (in the example shown, the first suction adapter device and the second suction adapter device correspond to a fourth embodiment of a suction adapter device 400 discussed in greater detail herein with respect to FIGS. 6A-6C).

As shown in FIGS. 2C-2G, the first suction adapter device 12A can be inserted directly into the suction tube connector 50 similarly to the example implementation shown in FIG. 2B. The first suction adapter device 12A can also couple with the first sheath portion 32 of the sheath 30 to connect the sheath 30 with the suction tube 40. In this example implementation, the first suction adapter device 12A couples with the first sheath portion 32 of the sheath 30 by insertion of a portion of the first suction adapter device 12A into the first sheath portion 32 of the sheath 30. Similarly, the second suction adapter device 12B couples with the second sheath portion 34 of the sheath 30 by insertion of a portion of the first suction adapter device 12A into the second sheath portion 34 of the sheath 30. In some embodiments, the second suction adapter device 12B can receive the first portion 22 of the medical tube 20 as shown in FIG. 2E. The suction generation device 60 can then be activated to draw the medical tube 20 through the sheath 30 until the first portion 22 of the medical tube 20 reaches the first suction adapter device 12A as shown in FIG. 2F.

FIGS. 2D-2F show further aspects of the system 10, including orientations of various components of the system 10 relative to one another. In particular, FIGS. 2D-2F show the medical tube 20 including the first portion 22 and the second portion 24, the sheath 30 including the first sheath portion 32 and the second sheath portion 34, and the suction tube 40 including the suction tube connector 50 positioned at the first portion 42 of the suction tube 40 and the suction generation device 60 positioned at a second portion 44 of the suction tube 40. As shown, the first sheath portion 32 of the sheath 30 includes a first sheath aperture 33 and the second sheath portion 34 of the sheath 30 includes a second sheath aperture 35 where the first sheath aperture 33 and the second sheath aperture 35 are connected by a sheath lumen 36. The suction tube 40 includes the suction tube connector 50; the suction tube connector 50 includes a first portion 52 defining the first connector aperture 53 and a second portion 54 defining a second aperture 55 where the first connector aperture 53 and the second aperture 55 are connected by a lumen 56. The second portion 54 of the suction tube connector 50 connects with or is otherwise integral to the first portion 42 of the suction tube 40 as shown. Similarly, the first portion 42 of the suction tube 40 defines a first suction tube aperture 43 and the second portion 44 of the suction tube 40 defines a second aperture 45 with the suction tube lumen 46 therebetween. As such, the first suction tube aperture 43 of the suction tube 40 connects with the second aperture 55 of the suction tube connector 50 such that the suction tube lumen 46 of the suction tube 40 communicates directly with the lumen 56 of the suction tube connector 50 as shown. The second portion 44 of the suction tube 40 connects with the suction generation device 60 to generate the suction force necessary to draw the medical tube 20 through the sheath 30 when the system 10 is assembled together as shown in FIGS. 2A-2F. Further, in some embodiments, the second portion 24 of the medical tube 20 can connect with a shunt valve 70 or another device prior to or following insertion of the first portion 22 of the medical tube 20 though the sheath 30, depending on the specific surgical application of the system 10.

As shown, the first suction adapter device 12A couples with the second sheath portion 34 of the sheath 30 at the second sheath aperture 35, and the second suction adapter device 12B couples with the first sheath portion 32 of the sheath 30 at the first sheath aperture 33. The first suction adapter device 12A also couples with the first portion 42 of the suction tube 40, which can be an indirect coupling (e.g., at the first portion 52 of the suction tube connector 50 in communication with the suction tube 40) or a direct coupling (e.g., coupling directly with the first portion 42 of the suction tube 40 absent the suction tube connector 50).

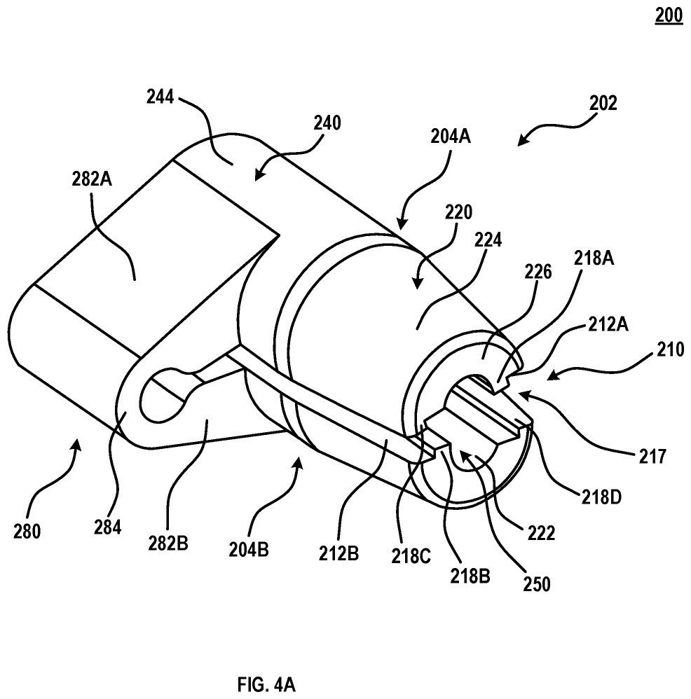

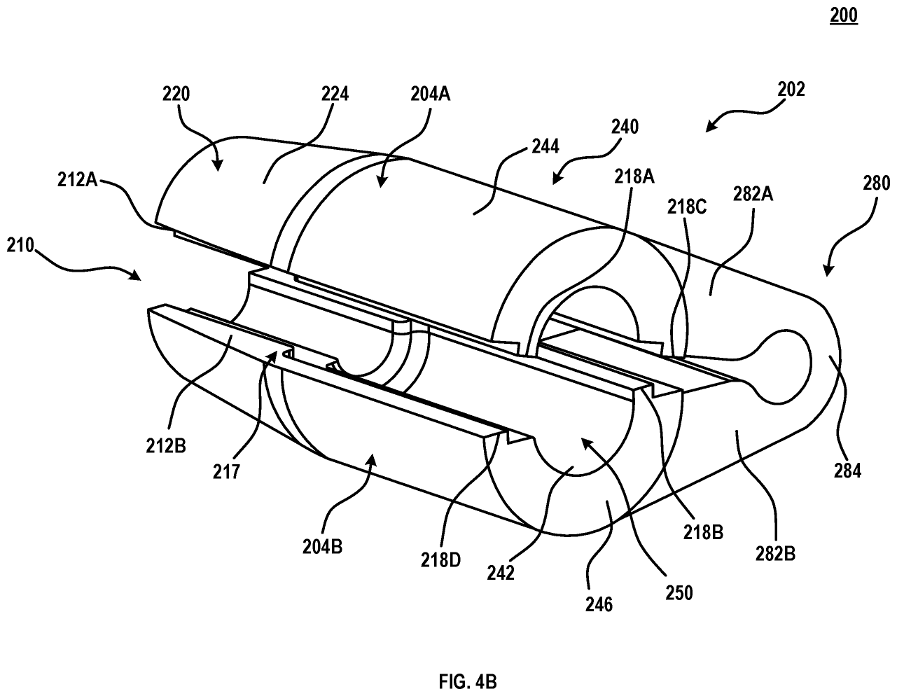

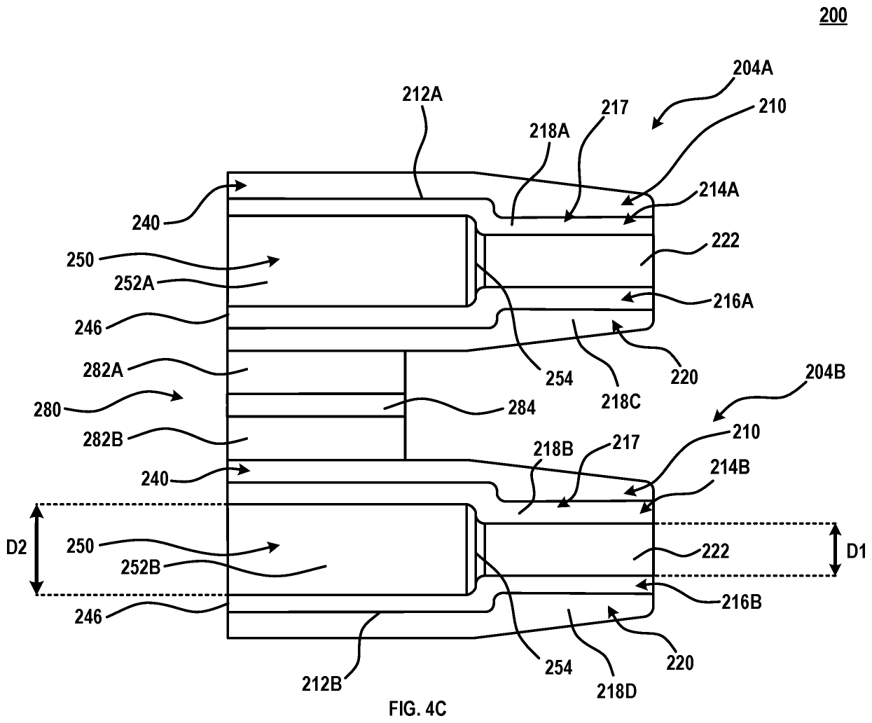

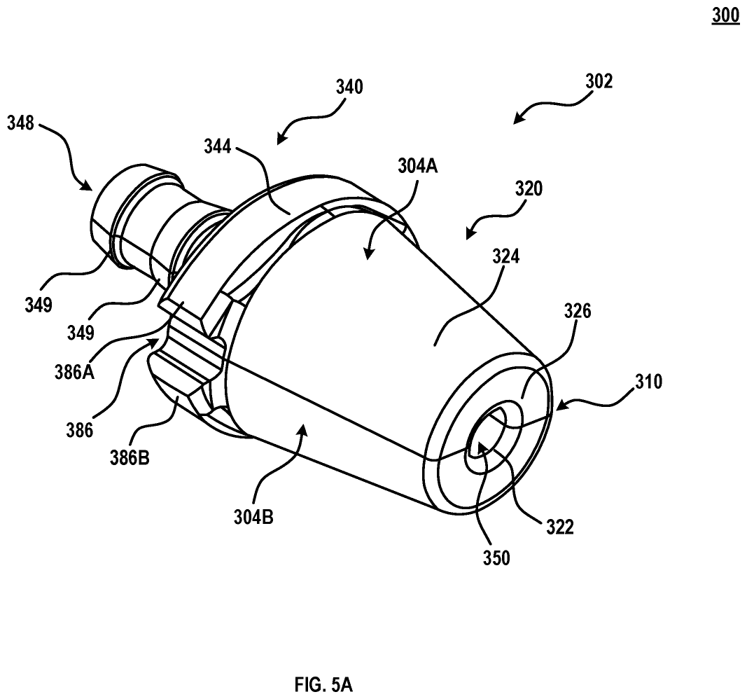

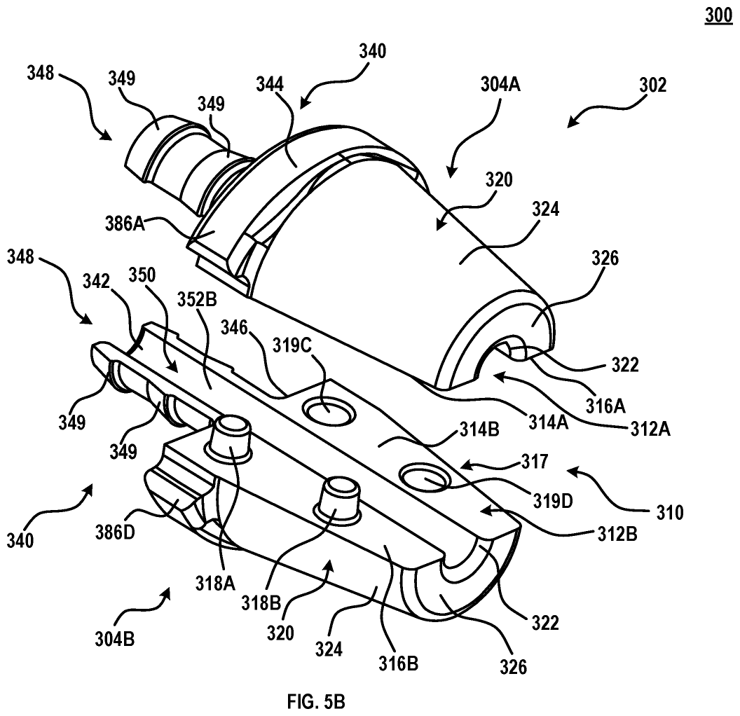

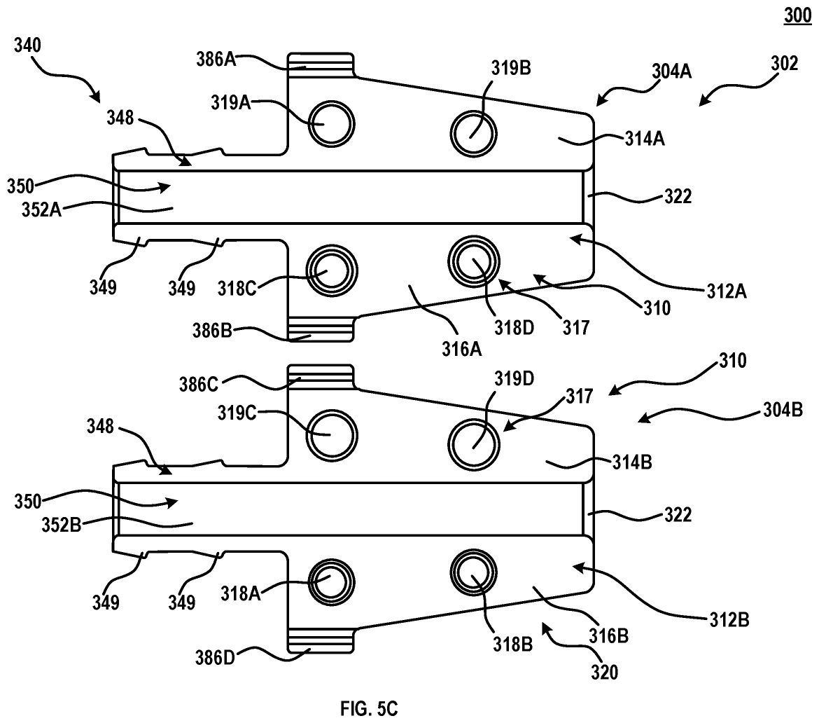

FIGS. 2E and 2F show engagement of the first suction adapter device 12A and the second suction adapter device 12B with the sheath 30 and the suction tube 40. As illustrated, the suction tube connector 50 includes an interior surface 58 of the lumen 56 that contacts the first suction adapter device 12A upon insertion of the first suction adapter device 12A into the first connector aperture 53 of the suction tube connector 50. Similarly, the sheath 30 includes an inner surface 38 of the sheath lumen 36 that contacts the first suction adapter device 12A upon insertion of the first suction adapter device 12A into the second sheath aperture 35 of the sheath 30 and that also contacts the second suction adapter device 12B upon insertion of the second suction adapter device 12B into the first sheath aperture 33 of the sheath 30. This is applicable for embodiments of the suction adapter device in which the suction adapter device is inserted into the sheath lumen 36 of the sheath 30, such as a third embodiment of a suction adapter device 300, a fourth embodiment of a suction adapter device 400, a fifth embodiment of a suction adapter device 500, or a sixth embodiment of a suction adapter device 600 discussed in greater detail herein with respect to FIGS. 5A-8C. For other embodiments such as a first embodiment of a suction adapter device 100 or a second embodiment of a suction adapter device 200 discussed in greater detail herein with respect to FIGS. 3A-4C, the suction adapter device can instead receive the sheath 30 by insertion of the sheath 30 into the suction adapter device as shown in FIG. 2B.

FIGS. 2E and 2F show positions of the medical tube 20 before and after being drawn through the sheath 30. As shown in FIG. 2E, the first portion 22 of the medical tube 20 can be inserted into the second suction adapter device 12B. The suction generation device 60 is then activated and generates the suction force that draws the first portion 22 of the medical tube 20 from the first sheath portion 32 of the sheath 30 to the second sheath portion 34 of the sheath 30. As shown in FIG. 2F, the first portion 22 of the medical tube 20 is then present at the second sheath portion 34 of the sheath 30 and the first suction adapter device 12A. FIG. 2G shows decoupling of the suction tube 40 (including the suction tube connector 50) from the first suction adapter device 12A by pulling the suction tube 40 in an axial direction away from the first suction adapter device 12A. Similarly, the first suction adapter device 12A can be decoupled from the first sheath portion 32 of the sheath 30 by pulling the first suction adapter device 12A in an axial direction away from the first sheath portion 32 of the sheath 30 as shown. Note that that a considerable length of the medical tube 20 (specifically, the second portion 24 of the medical tube 20) can remain external to the second sheath portion 34 of the sheath 30 as shown; in some embodiments, an external device such as the shunt valve 70 can already be connected to the second portion 24 of the medical tube 20. As such, it may not be advantageous to simply pull the second suction adapter device 12B away from the second sheath portion 34 of the sheath 30. As a result, the second suction adapter device 12B can be configured to open for decoupling from the medical tube 20. As such, to decouple the second suction adapter device 12B from the second sheath portion 34 of the sheath 30, the second suction adapter device 12B can be gently removed from the second sheath portion 34 of the sheath 30, with care taken so as not to damage the medical tube 20. A practitioner can then open the second suction adapter device 12B (e.g., lateral removal) to expose the medical tube 20 therein and decouple the second suction adapter device 12B from the medical tube as shown. In this manner, the second suction adapter device 12B allows lateral removal without decoupling devices such as the shunt valve 70 that may be present at the second portion 24 of the medical tube 20, and also provides opportunities for error correction. Note that the second suction adapter device 12B can be identical to the first suction adapter device 12A, and can be configured according to embodiments of the suction adapter device 100, 200, 300, 400, 500 and/or 600 further discussed herein.

### Suction Adapter Devices

FIGS. 3A-3C show a first embodiment of a suction adapter device 100 for implementation with the system 10. The suction adapter device 100 can be used interchangeably as the first suction adapter device 12A or the second suction adapter device 12B. As shown, the suction adapter device 100 includes a body 102 having a lumen 150 that establishes fluid-flow communication with various components of the system 10 discussed above with relation to FIGS. 1A-2G. As shown, the body 102 defines a nose portion 120 having a first aperture 122 that communicates with the lumen 150, and a sheath connection portion 140 having a second aperture 142 that communicates with the lumen 150. In the context of the system 10 shown in FIGS. 1A-2G, the nose portion 120 of the suction adapter device 100 (e.g., as the first suction adapter device 12A) is configured for engagement with the suction tube 40, and the sheath connection portion 140 of the suction adapter device 100 is configured for engagement with the sheath 30.

As shown, the body 102 including the nose portion 120, the sheath connection portion 140 and the lumen 150 can be divided into (or otherwise collectively defined by) a first section 104A and a second section 104B, where the first section 104A and the second section 104B are divided at a coupling portion 110 of the body 102. In the embodiment of FIGS. 3A-3C, the coupling portion 110 includes a slit 106 along the body 102 and communicating with the lumen 150 that separates a first face 112A of the first section 104A and a second face 112B of the second section 104B as shown. This enables a practitioner to open the suction adapter device 100 at the slit 106 and remove the medical tube 20 after passing the medical tube 20 through the sheath 30 during use of the system 10 as discussed above. Note that the coupling portion 110 does not necessarily need to lock the first section 104A and the second section 104B together; the circumferential force of the suction tube 40 when coupled with the suction adapter device 100 presses the first section 104A and the second section 104B together to prevent separation of the first section 104A from the second section 104B during operation.

Further, the lumen 150 can be divided between the first section 104A and the second section 104B; where the first section 104A includes a first lumen section 152A along the first face 112A and the second section 104B includes a second lumen section 152B along the second face 112B. For this embodiment, the first section 104A and the second section 104B can be non-removably connected with one another along one side of the lumen 150 and separated from one another at the slit 106 along the other side of the lumen 150. As such, the body 102 can include a hinge portion 180 at which the first section 104A and the second section 104B are non-removably connected opposite from the slit 106 as shown. The suction adapter device 100 can be made of a very soft rubber, silicon, or another suitable material to enable the hinge portion 180 to flex upon manual actuation of the first section 104A away from the second section 104B to access the lumen 150 therein. The suction adapter device 100 can include one or more grip protrusions 186 to aid in opening the device. In particular, the first section 104A can include a first grip protrusion 186A and the second section 104B can include a second grip protrusion 186B that provide points-of-contact for manual actuation of the first section 104A away from the second section 104B.

The nose portion 120 of the suction adapter device 100 is configured to couple with the suction tube 40 of the system 10 for connection with the suction generation device 60. In particular, as discussed above with reference to FIG. 2E, the suction adapter device 100 (as the first suction adapter device 12A) is configured to couple with the suction tube 40 at the suction tube connector 50 upon insertion of the suction adapter device 100 into the first connector aperture 53 of the suction tube connector 50. The nose portion 120 includes an outer conical surface 124 that couples with the interior surface 58 of the lumen 56 when inserted into the first connector aperture 53 of the suction tube connector 50. The outer conical surface 124 presses against the interior surface 58 to create a vacuum seal for drawing the medical tube 20 through the sheath 30 using the suction generation device 60, which communicates with the sheath 30 upon connection of the suction tube 40 with the sheath 30 by the suction adapter device 100. Further, as shown, the nose portion 120 includes a first terminal surface 126 that defines the first aperture 122 that communicates with the lumen 150.

The sheath connection portion 140 includes an outer surface 144 adjacent to the outer conical surface 124 of the nose portion 120. The outer surface 144 can be wide enough to provide a comfortable gripping area for a practitioner when handling the suction adapter device 100; in some embodiments, the first grip protrusion 186A of the first section 104A and the second grip protrusion 186B of the second section 104B can be present along the outer surface 144 of the sheath connection portion 140. The sheath connection portion 140 can further define a second terminal surface 146 that defines the second aperture 142 that communicates with the lumen 150. In the embodiment of FIGS. 3A-3C, the second aperture 142 of the sheath connection portion 140 is configured to receive the first sheath portion 32 or the second sheath portion 34 of the sheath 30. (e.g., the first sheath portion 32 or the second sheath portion 34 of the sheath 30 can be inserted into the second aperture 142 of the sheath connection portion 140).

As shown in FIG. 3C, and with additional reference to the system 10 of FIGS. 2A-2G, the first aperture 122 of the nose portion 120 of the suction adapter device 100 defines a first diameter D1 and the second aperture 142 of the sheath connection portion 140 of the suction adapter device 100 defines a second diameter D2. As such, the lumen 150 can be of the first diameter D1 towards the first aperture 122 of the nose portion 120 and the second diameter D2 towards the second aperture 142 of the sheath connection portion 140. The lumen 150 can include a tapering portion 154 that transitions the diameter of the lumen 150 from the first diameter D1 to the second diameter D2.

In one aspect, the first diameter D1 can be small enough to “catch” the medical tube at the suction adapter device 100 (as the first suction adapter device 12A) when the medical tube 20 is being drawn through the sheath 30 by the suction generation device 60. Further, the second diameter D2 can be large enough to receive the first sheath portion 32 or the second sheath portion 34 of the sheath 30 at the sheath connection portion 140 of the suction adapter device 100 (as the first suction adapter device 12A or the second suction adapter device 12B). During operation, after the medical tube 20 has been drawn through the sheath 30 and the first portion 22 of the medical tube 20 has arrived at the first sheath portion 32 of the sheath 30, the first portion 22 of the medical tube 20 remains within the lumen 150 of the suction adapter device 100 for lateral removal as discussed above with reference to FIGS. 2F and 2G (e.g., by separating the first section 104A from the second section 104B at the slit 106 and accessing the lumen 150 therein).

Continuing with FIG. 3C and in reference to the arrangement of the system 10 of FIG. 2E featuring the second suction adapter device 12B, the first diameter D1 can also be large enough for insertion of the first portion 22 of the medical tube 20 when the suction adapter device 100 is coupled with the second sheath portion 34 of the sheath 30 for threading of the medical tube 20 through the suction adapter device 100. Unlike the example shown in FIG. 2E, however, the suction adapter device 100 can similarly couple with the second sheath portion 34 of the sheath 30 at the sheath connection portion 140 by insertion of the second sheath portion 34 of the sheath 30 into the second aperture 142 of the suction adapter device 100 (as the second suction adapter device 12B) similar to the first suction adapter device 12A shown in FIGS. 2A and 2B.

FIGS. 4A-4C show a second embodiment of a suction adapter device 200 for implementation with the system 10. The suction adapter device 200 can be used interchangeably as the first suction adapter device 12A or the second suction adapter device 12B. As shown, the suction adapter device 200 includes a body 202 having a lumen 250 that establishes fluid-flow communication with various components of the system 10 discussed above with relation to FIGS. 1A-2G. As shown, the body 202 defines a nose portion 220 having a first aperture 222 that communicates with the lumen 250, and a sheath connection portion 240 having a second aperture 242 that communicates with the lumen 250. In the context of the system 10 shown in FIGS. 1A-2G, the nose portion 220 of the suction adapter device 200 (e.g., as the first suction adapter device 12A) is configured for engagement with the suction tube 40, and the sheath connection portion 240 of the suction adapter device 200 is configured for engagement with the sheath 30.

As shown, the body 202 including the nose portion 220, the sheath connection portion 240 and the lumen 250 can be divided into (or otherwise collectively defined by) a first section 204A and a second section 204B, where the first section 204A and the second section 204B are divided at a coupling portion 210 of the body 202. In the embodiment of FIGS. 4A-4C, the coupling portion 210 includes a first face 212A of the first section 204A and a second face 212B of the second section 204B as shown. This enables a practitioner to open the suction adapter device 200 by separating the first face 212A and the second face 212B to access the lumen 250 therebetween and remove the medical tube 20 after passing the medical tube 20 through the sheath 30 during use of the system 10 as discussed above. Note that the coupling portion 210 does not necessarily need to lock the first section 204A and the second section 204B together; the circumferential force of the suction tube 40 when coupled with the suction adapter device 200 presses the first section 204A and the second section 204B together to prevent separation of the first section 204A from the second section 204B during operation. In some embodiments, the coupling portion 210 defines a plurality of coupling elements 217 along the first face 212A of the first section 204A and the second face 212B of the second section 204B that aid in alignment of the first section 204A with the second section 204B. The first face 212A of the first section 204A can include a first half 214A and a second half 216A, where the first half 214A includes a first groove portion 218A of the plurality of coupling elements 217 and the second half 216A includes a second groove portion 218B of the plurality of coupling elements 217. Similarly, the second face 212B of the second section 204B can include a first half 214B and a second half 216B, where the first half 214B includes a third groove portion 218C of the plurality of coupling elements 217 and the second half 216B includes a fourth groove portion 218D of the plurality of coupling elements 217. When coupled together, the first groove portion 218A of the first face 212A and the fourth groove portion 218D of the second face 212B seat against one another. Similarly, when coupled together, the second groove portion 218B of the first face 212A and the third groove portion 218C of the second face 212B seat against one another.

Further, the lumen 250 can be divided between the first section 204A and the second section 204B; where the first section 204A includes a first lumen section 252A along the first face 212A (e.g., separating the first half 214A from the second half 216A of the first face 212A) and the second section 204B includes a second lumen section 252B along the second face 212B (e.g., separating the first half 214B from the second half 216B of the second face 212B).

To prevent loss of the first section 204A or the second section 204B, the body 202 can include a hinge portion 280 at which the first section 204A and the second section 204B are non-removably connected. The hinge portion 280 can be a “living hinge” arrangement, and the suction adapter device 200 can be made of a flexible plastic or another suitable material to enable the hinge portion 280 to flex when manually separating the first section 204A from the second section 204B to access the lumen 250 therein. As shown, the hinge portion 280 can include a first arm 282A associated with the first section 204A and a second arm 282B associated with the second section 204B; the first arm 282A and the second arm 282B can be connected at a hinge connector 284.

The nose portion 220 of the suction adapter device 200 is configured to couple with the suction tube 40 of the system 10 for connection with the suction generation device 60. In particular, as discussed above with reference to FIG. 2E, the suction adapter device 200 (as the first suction adapter device 12A) is configured to couple with the suction tube 40 at the suction tube connector 50 upon insertion of the suction adapter device 200 into the first connector aperture 53 of the suction tube connector 50. The nose portion 220 includes an outer conical surface 224 that couples with the interior surface 58 of the lumen 56 when inserted into the first connector aperture 53 of the suction tube connector 50. The outer conical surface 224 presses against the interior surface 58 to create a vacuum seal for drawing the medical tube 20 through the sheath 30 using the suction generation device 60, which communicates with the sheath 30 upon connection of the suction tube 40 with the sheath 30 by the suction adapter device 200. Further, as shown, the nose portion 220 includes a first terminal surface 226 that defines the first aperture 222 that communicates with the lumen 250.

The sheath connection portion 240 includes an outer surface 244 adjacent to the outer conical surface 224 of the nose portion 220. The outer surface 244 can be wide enough to provide a comfortable gripping area for a practitioner when handling the suction adapter device 200. The sheath connection portion 240 can further define a second terminal surface 246 that defines the second aperture 242 that communicates with the lumen 250. In the embodiment of FIGS. 4A-4C, the second aperture 242 of the sheath connection portion 240 is configured to receive the first sheath portion 32 or the second sheath portion 34 of the sheath 30. (e.g., the first sheath portion 32 or the second sheath portion 34 of the sheath 30 can be inserted into the second aperture 142 of the sheath connection portion 240).

As shown in FIG. 4C, and with additional reference to the system 10 of FIGS. 2A-2G, the first aperture 222 of the nose portion 220 of the suction adapter device 200 defines a first diameter D1 and the second aperture 242 of the sheath connection portion 240 of the suction adapter device 200 defines a second diameter D2. As such, the lumen 250 can be of the first diameter D1 towards the first aperture 222 of the nose portion 220 and the second diameter D2 towards the second aperture 242 of the sheath connection portion 240. The lumen 250 can include a tapering portion 254 that transitions the diameter of the lumen 250 from the first diameter D1 to the second diameter D2.

In one aspect, the first diameter D1 can be small enough to “catch” the medical tube at the suction adapter device 200 (as the first suction adapter device 12A) when the medical tube 20 is being drawn through the sheath 30 by the suction generation device 60. Further, the second diameter D2 can be large enough to receive the first sheath portion 32 or the second sheath portion 34 of the sheath 30 at the sheath connection portion 240 of the suction adapter device 200 (as the first suction adapter device 12A or the second suction adapter device 12B). During operation, after the medical tube 20 has been drawn through the sheath 30 and the first portion 22 of the medical tube 20 has arrived at the first sheath portion 32 of the sheath 30, the first portion 22 of the medical tube 20 remains within the lumen 250 of the suction adapter device 200 for lateral removal as discussed above with reference to FIGS. 2F and 2G (e.g., by separating the first section 204A from the second section 204B and accessing the lumen 250 therein).

Continuing with FIG. 4C and in reference to the arrangement of the system 10 of FIG. 2E featuring the second suction adapter device 12B, the first diameter D1 can also be large enough for insertion of the first portion 22 of the medical tube 20 when the suction adapter device 200 is coupled with the second sheath portion 34 of the sheath 30 for threading of the medical tube 20 through the suction adapter device 200. Unlike the example shown in FIG. 2E, however, the suction adapter device 200 can similarly couple with the second sheath portion 34 of the sheath 30 at the sheath connection portion 240 by insertion of the second sheath portion 34 of the sheath 30 into the second aperture 242 of the suction adapter device 200 (as the second suction adapter device 12B) similar to the first suction adapter device 12A shown in FIGS. 2A and 2B.

FIGS. 5A-5C show a third embodiment of a suction adapter device 300 for implementation with the system 10. The suction adapter device 300 can be used interchangeably as the first suction adapter device 12A or the second suction adapter device 12B. As shown, the suction adapter device 300 includes a body 302 having a lumen 350 that establishes fluid-flow communication with various components of the system 10 discussed above with relation to FIGS. 1A-2G. As shown, the body 302 defines a nose portion 320 having a first aperture 322 that communicates with the lumen 350, and a sheath connection portion 340 having a second aperture 342 that communicates with the lumen 350. In the context of the system 10 shown in FIGS. 1A-2G, the nose portion 320 of the suction adapter device 300 (e.g., as the first suction adapter device 12A) is configured for engagement with the suction tube 40, and the sheath connection portion 340 of the suction adapter device 300 is configured for engagement with the sheath 30.

As shown, the body 302 including the nose portion 320, the sheath connection portion 340 and the lumen 350 can be divided into (or otherwise collectively defined by) a first section 304A and a second section 304B, where the first section 304A and the second section 304B are divided at a coupling portion 310 of the body 302. In the embodiment of FIGS. 5A-5C, the coupling portion 310 includes a first face 312A of the first section 304A and a second face 312B of the second section 304B as shown. This enables a practitioner to open the suction adapter device 300 by separating the first face 312A and the second face 312B to access the lumen 350 therebetween and remove the medical tube 20 after passing the medical tube 20 through the sheath 30 during use of the system 10 as discussed above. Note that the coupling portion 310 does not necessarily need to lock the first section 304A and the second section 304B together; the circumferential force of the suction tube 40 and the sheath 30 when coupled with the suction adapter device 300 presses the first section 304A and the second section 304B together to prevent separation of the first section 304A from the second section 304B during operation. In some embodiments, the coupling portion 310 defines a plurality of coupling elements 317 along the first face 312A of the first section 304A and the second face 312B of the second section 304B that aid in alignment of the first section 304A with the second section 304B. The first face 312A of the first section 304A can include a first half 314A and a second half 316A, where the first half 314A includes one or more recesses including a first recess 319A and a second recess 319B of the plurality of coupling elements 317 and the second half 316A includes one or more pins including a third pin 318C and a fourth pin 318D of the plurality of coupling elements 317. Similarly, the second face 312B of the second section 304B can include a first half 314B and a second half 316B, where the first half 314B includes one or more recesses including a third recess 319C and a fourth recess 319D of the plurality of coupling elements 317 and the second half 316B includes one or more pins including a first pin 318A and a second pin 318B of the plurality of coupling elements 317. When coupled together, the first recess 319A and the second recess 319B of the first face 312A respectively couple with the first pin 318A and the second pin 318B of the second face 312B. Similarly, when coupled together, the third recess 319C and the fourth recess 319D of the second face 312B respectively couple with the third pin 318C and the fourth pin 318D of the first face 312A.

Further, the lumen 350 can be divided between the first section 304A and the second section 304B; where the first section 304A includes a first lumen section 352A along the first face 312A (e.g., separating the first half 314A from the second half 316A of the first face 312A) and the second section 304B includes a second lumen section 352B along the second face 312B (e.g., separating the first half 314B from the second half 316B of the second face 312B).

The suction adapter device 300 can include one or more grip protrusions 386 to aid in opening the device. In particular, the first section 304A can include a first grip protrusion 386A and a second grip protrusion 386B and the second section 304B can include a third grip protrusion 386C and a fourth grip protrusion 386D that provide points-of-contact for manual actuation of the first section 304A away from the second section 304B.

The nose portion 320 of the suction adapter device 300 is configured to couple with the suction tube 40 of the system 10 for connection with the suction generation device 60. In particular, as discussed above with reference to FIG. 2E, the suction adapter device 300 (as the first suction adapter device 12A) is configured to couple with the suction tube 40 at the suction tube connector 50 upon insertion of the suction adapter device 300 into the first connector aperture 53 of the suction tube connector 50. The nose portion 320 includes an outer conical surface 324 that couples with the interior surface 58 of the lumen 56 when inserted into the first connector aperture 53 of the suction tube connector 50. The outer conical surface 324 presses against the interior surface 58 to create a vacuum seal for drawing the medical tube 20 through the sheath 30 using the suction generation device 60, which communicates with the sheath 30 upon connection of the suction tube 40 with the sheath 30 by the suction adapter device 300. Further, as shown, the nose portion 320 includes a first terminal surface 326 that defines the first aperture 322 that communicates with the lumen 350.

The sheath connection portion 340 includes an outer surface 344 adjacent to the outer conical surface 324 of the nose portion 320. The outer surface 344 can be wide enough to provide a comfortable gripping area for a practitioner when handling the suction adapter device 300; in some embodiments, the one or more grip protrusions 386 of the first section 304A and the second section 304B can be present along the outer surface 344 of the sheath connection portion 340. The sheath connection portion 340 can further define a second terminal surface 346 having a protrusion 348 extending along the direction of elongation of the lumen 350. As shown, the protrusion 348 defines the second aperture 342 that communicates with the lumen 350, and can optionally include one or more barbs 349. In the embodiment of FIGS. 5A-5C, the protrusion 348 of the sheath connection portion 340 is configured for insertion into the first sheath portion 32 or the second sheath portion 34 of the sheath 30 (e.g., the first sheath aperture 33 of the first sheath portion 32 or the second sheath aperture 35 of the second sheath portion 34 of the sheath 30 can receive the protrusion 348 of the sheath connection portion 340) to establish fluid flow communication between the sheath lumen 36 of the sheath 30 and the lumen 350 of the suction adapter device 300. Further, the protrusion 348 can be secured within the sheath lumen 36 of the sheath 30 through friction-fit contact with the inner surface 38 of the sheath 30; in some embodiments, the one or more barbs 349 aid in maintaining this connection. The second terminal surface 346 provides an abutment surface for the terminal end of the first sheath portion 32 or the second sheath portion 34 of the sheath 30.

As shown in FIG. 5C, and with additional reference to the system 10 of FIGS. 2A-2G, the first aperture 322 of the nose portion 320 of the suction adapter device 300 defines a first diameter D1 and the second aperture 342 of the sheath connection portion 340 of the suction adapter device 300 defines a second diameter D2. As such, the lumen 350 can be of the first diameter D1 towards the first aperture 322 of the nose portion 320 and the second diameter D2 towards the second aperture 342 of the sheath connection portion 240; in the embodiment shown, the first diameter D1 can be equal to the second diameter D2.

In one aspect, the first diameter D1 can be small enough to “catch” the medical tube at the suction adapter device 200 (as the first suction adapter device 12A) when the medical tube 20 is being drawn through the sheath 30 by the suction generation device 60. Further, the second diameter D2 and the protrusion 348 can be small enough such that they can be fully encapsulated within the first sheath portion 32 or the second sheath portion 34 of the sheath 30 at the sheath connection portion 340 of the suction adapter device 300 (as the first suction adapter device 12A or the second suction adapter device 12B). During operation, after the medical tube 20 has been drawn through the sheath 30 and the first portion 22 of the medical tube 20 has arrived at the first sheath portion 32 of the sheath 30, the first portion 22 of the medical tube 20 remains within the lumen 350 of the suction adapter device 300 for lateral removal as discussed above with reference to FIGS. 2F and 2G (e.g., by separating the first section 304A from the second section 304B and accessing the lumen 350 therein).

Continuing with FIG. 5C and in reference to the arrangement of the system 10 of FIG. 2E featuring the second suction adapter device 12B, the first diameter D1 can also be large enough for insertion of the first portion 22 of the medical tube 20 when the suction adapter device 300 is coupled with the second sheath portion 34 of the sheath 30 for threading of the medical tube 20 through the suction adapter device 300.

FIGS. 6A-6C show a fourth embodiment of a suction adapter device 400 for implementation with the system 10. The suction adapter device 400 can be used interchangeably as the first suction adapter device 12A or the second suction adapter device 12B. As shown, the suction adapter device 400 includes a body 402 having a lumen 450 that establishes fluid-flow communication with various components of the system 10 discussed above with relation to FIGS. 1A-2G. As shown, the body 402 defines a nose portion 420 having a first aperture 422 that communicates with the lumen 450, and a sheath connection portion 440 having a second aperture 442 that communicates with the lumen 450. In the context of the system 10 shown in FIGS. 1A-2G, the nose portion 420 of the suction adapter device 400 (e.g., as the first suction adapter device 12A) is configured for engagement with the suction tube 40, and the sheath connection portion 440 of the suction adapter device 400 is configured for engagement with the sheath 30. Notably, the suction adapter device 400 differs from previously-discussed embodiments by inclusion of a funnel 428 at the first aperture 422 that aids insertion of the medical tube 20 as discussed above with reference to FIG. 2E.

As shown, the body 402 including the nose portion 420, the sheath connection portion 440 and the lumen 450 can be divided into (or otherwise collectively defined by) a first section 404A and a second section 404B, where the first section 404A and the second section 404B are divided at a coupling portion 410 of the body 402. In the embodiment of FIGS. 6A-6C, the coupling portion 410 includes a first face 412A of the first section 404A and a second face 412B of the second section 404B as shown. This enables a practitioner to open the suction adapter device 400 by separating the first face 412A and the second face 412B to access the lumen 450 therebetween and remove the medical tube 20 after passing the medical tube 20 through the sheath 30 during use of the system 10 as discussed above. Note that the coupling portion 410 does not necessarily need to lock the first section 404A and the second section 404B together; the circumferential force of the suction tube 40 and the sheath 30 when coupled with the suction adapter device 400 presses the first section 404A and the second section 404B together to prevent separation of the first section 404A from the second section 404B during operation. In some embodiments, the coupling portion 410 defines a plurality of coupling elements 417 along the first face 412A of the first section 404A and the second face 412B of the second section 404B that aid in alignment of the first section 404A with the second section 404B. The first face 412A of the first section 404A can include a first half 414A and a second half 416A, where the first half 414A includes one or more recesses including a first recess 419A and a second recess 419B of the plurality of coupling elements 417 and the second half 416A includes one or more pins including a third pin 418C and a fourth pin 418D of the plurality of coupling elements 417. Similarly, the second face 412B of the second section 404B can include a first half 414B and a second half 416B, where the first half 414B includes one or more recesses including a third recess 419C and a fourth recess 419D of the plurality of coupling elements 417 and the second half 416B includes one or more pins including a first pin 418A and a second pin 418B of the plurality of coupling elements 417. When coupled together, the first recess 419A and the second recess 419B of the first face 412A respectively couple with the first pin 418A and the second pin 418B of the second face 412B. Similarly, when coupled together, the third recess 419C and the fourth recess 419D of the second face 412B respectively couple with the third pin 418C and the fourth pin 418D of the first face 412A.

Further, the lumen 450 can be divided between the first section 404A and the second section 404B; where the first section 404A includes a first lumen section 452A along the first face 412A (e.g., separating the first half 414A from the second half 416A of the first face 412A) and the second section 404B includes a second lumen section 452B along the second face 412B (e.g., separating the first half 414B from the second half 416B of the second face 412B).

The suction adapter device 400 can include one or more grip protrusions 486 to aid in opening the device. In particular, the first section 404A can include a first grip protrusion 486A and a second grip protrusion 486B and the second section 404B can include a third grip protrusion 486C and a fourth grip protrusion 486D that provide points-of-contact for manual actuation of the first section 404A away from the second section 404B.

The nose portion 420 of the suction adapter device 400 is configured to couple with the suction tube 40 of the system 10 for connection with the suction generation device 60. In particular, as discussed above with reference to FIG. 2E, the suction adapter device 400 (as the first suction adapter device 12A) is configured to couple with the suction tube 40 at the suction tube connector 50 upon insertion of the suction adapter device 400 into the first connector aperture 53 of the suction tube connector 50. The nose portion 420 includes an outer conical surface 424 that couples with the interior surface 58 of the lumen 56 when inserted into the first connector aperture 53 of the suction tube connector 50. The outer conical surface 424 presses against the interior surface 58 to create a vacuum seal for drawing the medical tube 20 through the sheath 30 using the suction generation device 60, which communicates with the sheath 30 upon connection of the suction tube 40 with the sheath 30 by the suction adapter device 400. In contrast with previous embodiments, the nose portion 420 of the suction adapter device 400 can include the first aperture 422 that is significantly wider than previous embodiments, and includes the funnel 428 that tapers into the lumen 450 as shown. The funnel 428 helps guide the medical tube 20 into the lumen 450 when positioned at the second sheath portion 34 of the sheath 30 as shown in the arrangement of FIG. 2E (with the suction adapter device 400 being the second suction adapter device 12B).

The sheath connection portion 440 includes an outer surface 444 adjacent to the outer conical surface 424 of the nose portion 420. The outer surface 444 can be wide enough to provide a comfortable gripping area for a practitioner when handling the suction adapter device 400; in some embodiments, the one or more grip protrusions 486 of the first section 404A and the second section 404B can be present along the outer surface 444 of the sheath connection portion 440. The sheath connection portion 440 can further define a second terminal surface 446 having a protrusion 448 extending along the direction of elongation of the lumen 450. As shown, the protrusion 448 defines the second aperture 442 that communicates with the lumen 450, and can optionally include one or more barbs 449. In the embodiment of FIGS. 6A-6C, the protrusion 448 of the sheath connection portion 440 is configured for insertion into the first sheath portion 32 or the second sheath portion 34 of the sheath 30 (e.g., the first sheath aperture 33 of the first sheath portion 32 or the second sheath aperture 35 of the second sheath portion 34 of the sheath 30 can receive the protrusion 448 of the sheath connection portion 440) to establish fluid flow communication between the sheath lumen 36 of the sheath 30 and the lumen 450 of the suction adapter device 400. Further, the protrusion 448 can be secured within the sheath lumen 36 of the sheath 30 through friction-fit contact with the inner surface 38 of the sheath 30; in some embodiments, the one or more barbs 449 aid in maintaining this connection. The second terminal surface 446 provides an abutment surface for the terminal end of the first sheath portion 32 or the second sheath portion 34 of the sheath 30.

As shown in FIG. 6C, and with additional reference to the system 10 of FIGS. 2A-2G, the first aperture 422 of the nose portion 420 of the suction adapter device 400 defines a first diameter D1 and the second aperture 442 of the sheath connection portion 440 of the suction adapter device 400 defines a second diameter D2. As such, the lumen 450 can be of the first diameter D1 towards the first aperture 422 of the nose portion 420 and the second diameter D2 towards the second aperture 442 of the sheath connection portion 440. In the embodiment shown, the first diameter D1 can be significantly greater than the second diameter D2, as the first diameter D1 needs to accommodate the funnel 428 of the nose portion 420 and the second diameter D2 and the sheath connection portion 440 need to be small enough for insertion into the sheath lumen 36 of the sheath 30. As such, the lumen 450 can include a tapering portion 454 that transitions the diameter of the lumen 450 from the first diameter D1 to the second diameter D2.

During operation, after the medical tube 20 has been drawn through the sheath 30 and the first portion 22 of the medical tube 20 has arrived at the first sheath portion 32 of the sheath 30, the first portion 22 of the medical tube 20 remains within the lumen 450 of the suction adapter device 400 for lateral removal as discussed above with reference to FIGS. 2F and 2G (e.g., by separating the first section 404A from the second section 404B and accessing the lumen 450 therein).

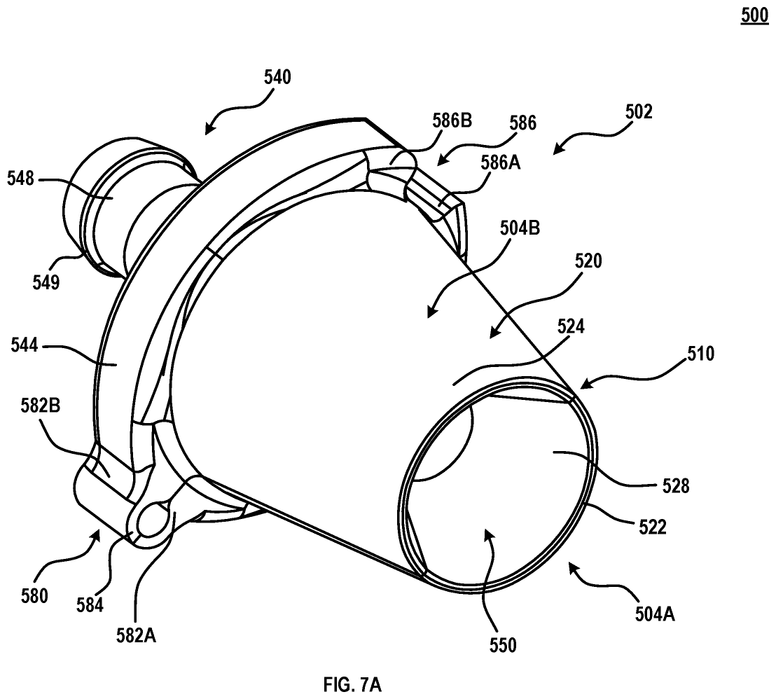

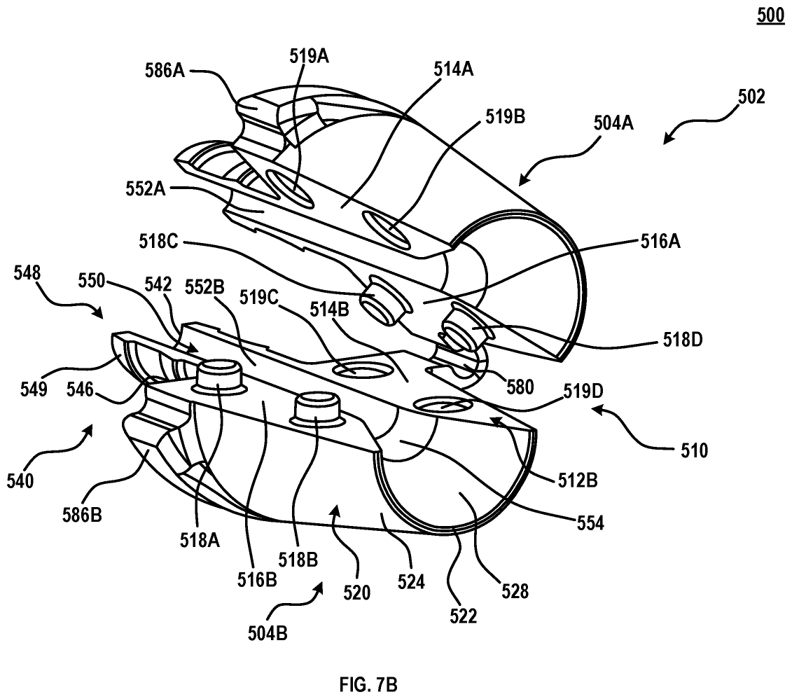

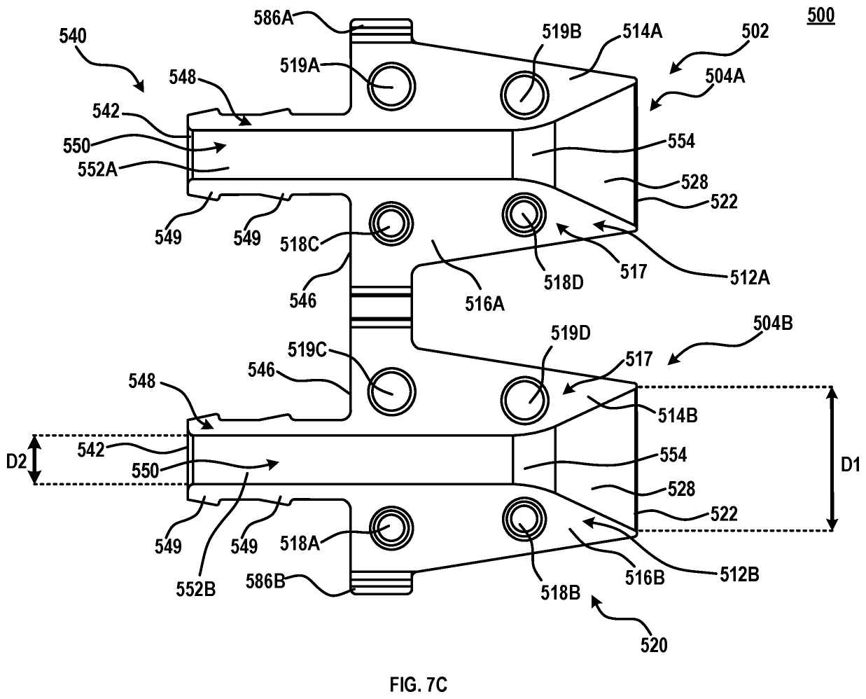

FIGS. 7A-7C show a fifth embodiment of a suction adapter device 500 for implementation with the system 10. The suction adapter device 500 can be used interchangeably as the first suction adapter device 12A or the second suction adapter device 12B. As shown, the suction adapter device 500 includes a body 502 having a lumen 550 that establishes fluid-flow communication with various components of the system 10 discussed above with relation to FIGS. 1A-2G. As shown, the body 502 defines a nose portion 520 having a first aperture 522 that communicates with the lumen 550, and a sheath connection portion 540 having a second aperture 542 that communicates with the lumen 550. In the context of the system 10 shown in FIGS. 1A-2G, the nose portion 520 of the suction adapter device 500 (e.g., as the first suction adapter device 12A) is configured for engagement with the suction tube 40, and the sheath connection portion 540 of the suction adapter device 500 is configured for engagement with the sheath 30. The suction adapter device 500 can be similar to the previously discussed suction adapter device 400 (of FIGS. 6A-6C) and can further include a hinge portion 580 that connects two discrete sections of the body 502 as will be discussed in greater detail.

As shown, the body 502 including the nose portion 520, the sheath connection portion 540 and the lumen 550 can be divided into (or otherwise collectively defined by) a first section 504A and a second section 504B, where the first section 504A and the second section 504B are divided at a coupling portion 510 of the body 502. In the embodiment of FIGS. 7A-7C, the coupling portion 510 includes a first face 512A of the first section 504A and a second face 512B of the second section 504B as shown. This enables a practitioner to open the suction adapter device 500 by separating the first face 512A and the second face 512B to access the lumen 550 therebetween and remove the medical tube 20 after passing the medical tube 20 through the sheath 30 during use of the system 10 as discussed above. Note that the coupling portion 510 does not necessarily need to lock the first section 504A and the second section 504B together; the circumferential force of the suction tube 40 and the sheath 30 when coupled with the suction adapter device 500 presses the first section 504A and the second section 504B together to prevent separation of the first section 504A from the second section 504B during operation. In some embodiments, the coupling portion 510 defines a plurality of coupling elements 517 along the first face 512A of the first section 504A and the second face 512B of the second section 504B that aid in alignment of the first section 504A with the second section 504B. The first face 512A of the first section 504A can include a first half 514A and a second half 516A, where the first half 514A includes one or more recesses including a first recess 519A and a second recess 519B of the plurality of coupling elements 517 and the second half 516A includes one or more pins including a third pin 518C and a fourth pin 518D of the plurality of coupling elements 517. Similarly, the second face 512B of the second section 504B can include a first half 514B and a second half 516B, where the first half 514B includes one or more recesses including a third recess 519C and a fourth recess 519D of the plurality of coupling elements 517 and the second half 516B includes one or more pins including a first pin 518A and a second pin 518B of the plurality of coupling elements 517. When coupled together, the first recess 519A and the second recess 519B of the first face 512A respectively couple with the first pin 518A and the second pin 518B of the second face 512B. Similarly, when coupled together, the third recess 519C and the fourth recess 519D of the second face 512B respectively couple with the third pin 518C and the fourth pin 518D of the first face 512A.

Further, the lumen 550 can be divided between the first section 504A and the second section 504B; where the first section 504A includes a first lumen section 552A along the first face 512A (e.g., separating the first half 514A from the second half 516A of the first face 512A) and the second section 504B includes a second lumen section 552B along the second face 512B (e.g., separating the first half 514B from the second half 516B of the second face 512B).

The suction adapter device 500 can include one or more grip protrusions 586 to aid in opening the device. In particular, the first section 504A can include a first grip protrusion 586A and the second section 504B can include a second grip protrusion 586B that provide points-of-contact for manual actuation of the first section 504A away from the second section 504B.

To prevent loss of the first section 504A or the second section 504B, the body 502 can include the hinge portion 580 at which the first section 504A and the second section 504B are non-removably connected. The hinge portion 580 can be a “living hinge” arrangement, and the suction adapter device 500 can be made of a flexible plastic or another suitable material to enable the hinge portion 580 to flex when manually separating the first section 504A from the second section 504B to access the lumen 550 therein. As shown, the hinge portion 580 can include a first arm 582A associated with the first section 504A and a second arm 582B associated with the second section 504B; the first arm 582A and the second arm 582B can be connected at a hinge connector 584.

The nose portion 520 of the suction adapter device 500 is configured to couple with the suction tube 40 of the system 10 for connection with the suction generation device 60. In particular, as discussed above with reference to FIG. 2E, the suction adapter device 500 (as the first suction adapter device 12A) is configured to couple with the suction tube 40 at the suction tube connector 50 upon insertion of the suction adapter device 500 into the first connector aperture 53 of the suction tube connector 50. The nose portion 520 includes an outer conical surface 524 that couples with the interior surface 58 of the lumen 56 when inserted into the first connector aperture 53 of the suction tube connector 50. The outer conical surface 524 presses against the interior surface 58 to create a vacuum seal for drawing the medical tube 20 through the sheath 30 using the suction generation device 60, which communicates with the sheath 30 upon connection of the suction tube 40 with the sheath 30 by the suction adapter device 500. Similar to the previous embodiments shown above with reference to FIGS. 6A-6C, the nose portion 520 of the suction adapter device 500 of FIGS. 7A-7C can include the first aperture 522 that is significantly wider and includes a funnel 528 that tapers into the lumen 550 as shown. The funnel 528 helps guide the medical tube 20 into the lumen 550 when positioned at the second sheath portion 34 of the sheath 30 as shown in the arrangement of FIG. 2E (with the suction adapter device 500 being the second suction adapter device 12B).

The sheath connection portion 540 includes an outer surface 544 adjacent to the outer conical surface 524 of the nose portion 520. The outer surface 544 can be wide enough to provide a comfortable gripping area for a practitioner when handling the suction adapter device 500; in some embodiments, the first grip protrusion 586A of the first section 504A and the second grip protrusion 586B of the second section 504B can be present along the outer surface 544 of the sheath connection portion 540. The sheath connection portion 540 can further define a second terminal surface 546 having a protrusion 548 extending along the direction of elongation of the lumen 550. As shown, the protrusion 548 defines the second aperture 542 that communicates with the lumen 550, and can optionally include one or more barbs 549. In the embodiment of FIGS. 7A-7C, the protrusion 548 of the sheath connection portion 540 is configured for insertion into the first sheath portion 32 or the second sheath portion 34 of the sheath 30 (e.g., the first sheath aperture 33 of the first sheath portion 32 or the second sheath aperture 35 of the second sheath portion 34 of the sheath 30 can receive the protrusion 548 of the sheath connection portion 540) to establish fluid flow communication between the sheath lumen 36 of the sheath 30 and the lumen 550 of the suction adapter device 500. Further, the protrusion 548 can be secured within the sheath lumen 36 of the sheath 30 through friction-fit contact with the inner surface 38 of the sheath 30; in some embodiments, the one or more barbs 549 aid in maintaining this connection. The second terminal surface 546 provides an abutment surface for the terminal end of the first sheath portion 32 or the second sheath portion 34 of the sheath 30.

As shown in FIG. 7C, and with additional reference to the system 10 of FIGS. 2A-2G, the first aperture 522 of the nose portion 520 of the suction adapter device 500 defines a first diameter D1 and the second aperture 542 of the sheath connection portion 540 of the suction adapter device 500 defines a second diameter D2. As such, the lumen 550 can be of the first diameter D1 towards the first aperture 522 of the nose portion 520 and the second diameter D2 towards the second aperture 542 of the sheath connection portion 540. In the embodiment shown, the first diameter D1 can be significantly greater than the second diameter D2, as the first diameter D1 needs to accommodate the funnel 528 of the nose portion 520 and the second diameter D2 and the sheath connection portion 540 need to be small enough for insertion into the sheath lumen 36 of the sheath 30. As such, the lumen 550 can include a tapering portion 554 that transitions the diameter of the lumen 550 from the first diameter D1 to the second diameter D2.

During operation, after the medical tube 20 has been drawn through the sheath 30 and the first portion 22 of the medical tube 20 has arrived at the first sheath portion 32 of the sheath 30, the first portion 22 of the medical tube 20 remains within the lumen 550 of the suction adapter device 500 for lateral removal as discussed above with reference to FIGS. 2F and 2G (e.g., by separating the first section 504A from the second section 504B and accessing the lumen 550 therein).

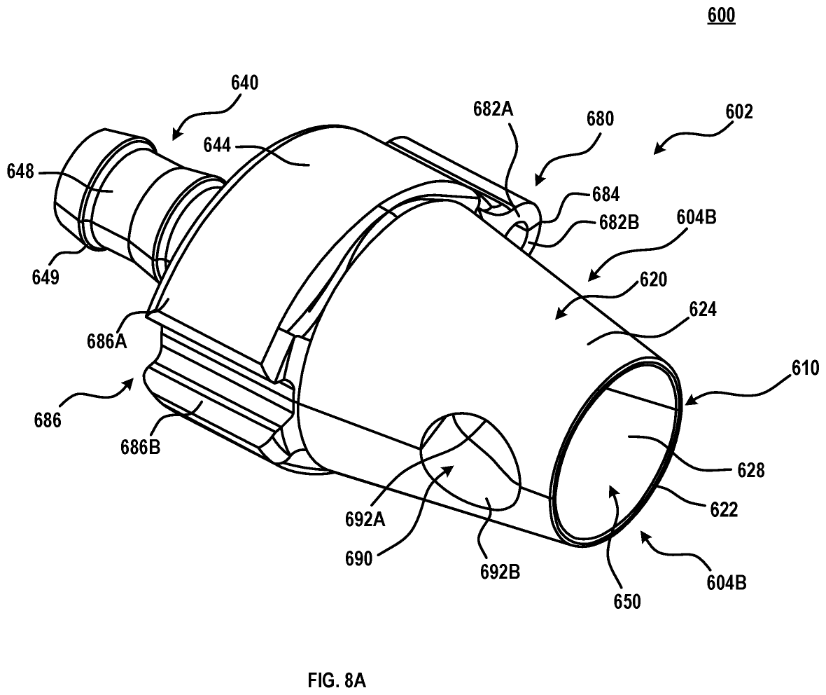

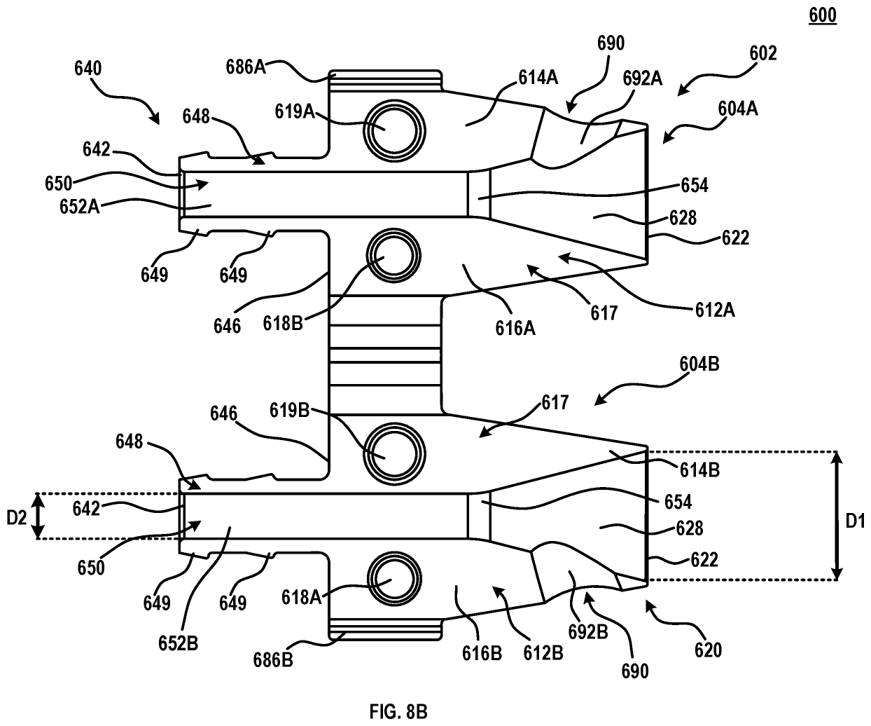

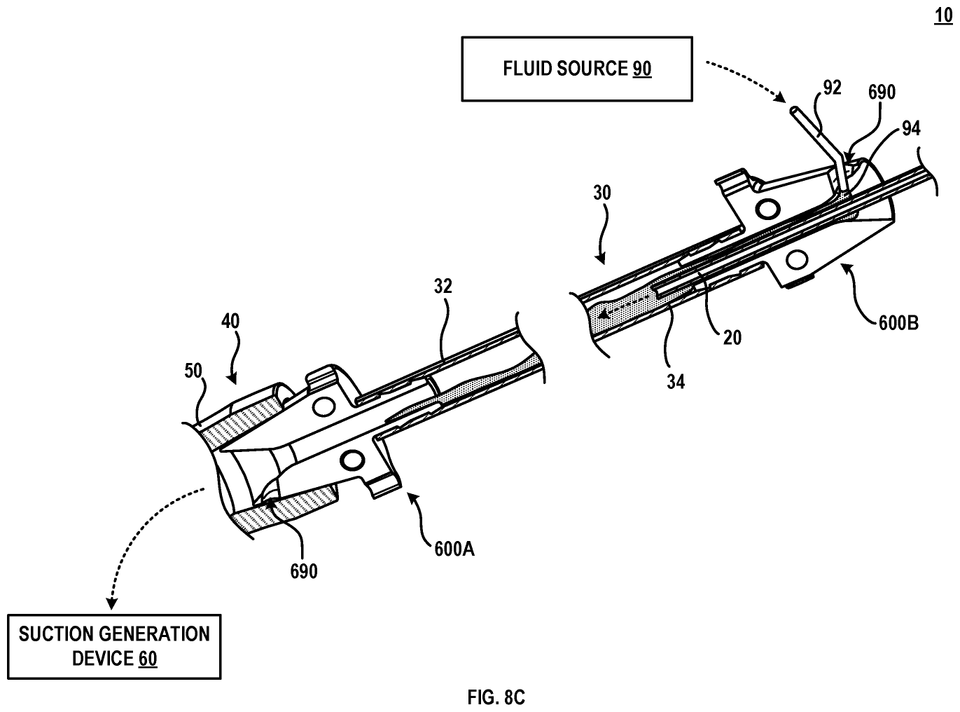

FIGS. 8A-8C show a sixth embodiment of a suction adapter device 600 for implementation with the system 10. The suction adapter device 600 can be used interchangeably as the first suction adapter device 12A or the second suction adapter device 12B. As shown, the suction adapter device 600 includes a body 602 having a lumen 650 that establishes fluid-flow communication with various components of the system 10 discussed above with relation to FIGS. 1A-2G. As shown, the body 602 defines a nose portion 620 having a first aperture 622 that communicates with the lumen 650, and a sheath connection portion 640 having a second aperture 642 that communicates with the lumen 650. In the context of the system 10 shown in FIGS. 1A-2G, the nose portion 620 of the suction adapter device 600 (e.g., as the first suction adapter device 12A) is configured for engagement with the suction tube 40, and the sheath connection portion 640 of the suction adapter device 600 is configured for engagement with the sheath 30. The suction adapter device 600 can be similar to the previously discussed suction adapter device 500 (of FIGS. 7A-7C) and can further include a fluid injection port 690 along the nose portion 620 that enables injection of a fluid such as saline into the sheath 30 to lubricate the interior of the sheath 30 as the medical tube 20 is being drawn through the sheath 30.

As shown, the body 602 including the nose portion 620, the sheath connection portion 640 and the lumen 650 can be divided into (or otherwise collectively defined by) a first section 604A and a second section 604B, where the first section 604A and the second section 604B are divided at a coupling portion 610 of the body 602. In the embodiment of FIGS. 8A-8C, the coupling portion 610 includes a first face 612A of the first section 604A and a second face 612B of the second section 604B as shown. This enables a practitioner to open the suction adapter device 600 by separating the first face 612A and the second face 612B to access the lumen 650 therebetween and remove the medical tube 20 after passing the medical tube 20 through the sheath 30 during use of the system 10 as discussed above. Note that the coupling portion 610 does not necessarily need to lock the first section 604A and the second section 604B together; the circumferential force of the suction tube 40 and the sheath 30 when coupled with the suction adapter device 600 presses the first section 604A and the second section 604B together to prevent separation of the first section 604A from the second section 604B during operation. In some embodiments, the coupling portion 610 defines a plurality of coupling elements 617 along the first face 612A of the first section 604A and the second face 612B of the second section 604B that aid in alignment of the first section 604A with the second section 604B. The first face 612A of the first section 604A can include a first half 614A and a second half 616A, where the first half 514A includes one or more recesses including a first recess 619A of the plurality of coupling elements 617 and the second half 616A includes one or more pins including a second pin 618B of the plurality of coupling elements 617. Similarly, the second face 612B of the second section 604B can include a first half 614B and a second half 616B, where the first half 614B includes one or more recesses including a second recess 619B of the plurality of coupling elements 617 and the second half 616B includes one or more pins including a first pin 618A of the plurality of coupling elements 617. When coupled together, the first recess 619A of the first face 612A couples with the first pin 618A of the second face 512B. Similarly, when coupled together, the second recess 619B of the second face 512B couples with the second pin 618B of the first face 612A.

Further, the lumen 650 can be divided between the first section 604A and the second section 604B; where the first section 604A includes a first lumen section 652A along the first face 612A (e.g., separating the first half 614A from the second half 616A of the first face 612A) and the second section 604B includes a second lumen section 652B along the second face 612B (e.g., separating the first half 614B from the second half 616B of the second face 612B).

The suction adapter device 600 can include one or more grip protrusions 686 to aid in opening the device. In particular, the first section 604A can include a first grip protrusion 686A and the second section 604B can include a second grip protrusion 686B that provide points-of-contact for manual actuation of the first section 604A away from the second section 604B.

To prevent loss of the first section 604A or the second section 604B, the body 602 can include a hinge portion 680 at which the first section 604A and the second section 604B are non-removably connected. The hinge portion 680 can be a “living hinge” arrangement, and the suction adapter device 600 can be made of a flexible plastic or another suitable material to enable the hinge portion 680 to flex when manually separating the first section 604A from the second section 604B to access the lumen 650 therein. As shown, the hinge portion 680 can include a first arm 682A associated with the first section 604A and a second arm 682B associated with the second section 604B; the first arm 682A and the second arm 682B can be connected at a hinge connector 684.

The nose portion 620 of the suction adapter device 600 is configured to couple with the suction tube 40 of the system 10 for connection with the suction generation device 60. In particular, as discussed above with reference to FIG. 2E, the suction adapter device 600 (as the first suction adapter device 12A) is configured to couple with the suction tube 40 at the suction tube connector 50 upon insertion of the suction adapter device 600 into the first connector aperture 53 of the suction tube connector 50. The nose portion 620 includes an outer conical surface 624 that couples with the interior surface 58 of the lumen 56 when inserted into the first connector aperture 53 of the suction tube connector 50. The outer conical surface 624 presses against the interior surface 58 to create a vacuum seal for drawing the medical tube 20 through the sheath 30 using the suction generation device 60, which communicates with the sheath 30 upon connection of the suction tube 40 with the sheath 30 by the suction adapter device 600. Similar to the previous embodiments shown above with reference to FIGS. 7A-7C, the nose portion 620 of the suction adapter device 600 of FIGS. 8A-8C can include the first aperture 622 that is significantly wider and includes a funnel 628 that tapers into the lumen 650 as shown. The funnel 628 helps guide the medical tube 20 into the lumen 650 when positioned at the second sheath portion 34 of the sheath 30 as shown in the arrangement of FIGS. 2E and 8C (with the suction adapter device 600 being the second suction adapter device 12B).

As further shown, the nose portion 620 includes a fluid injection port 690 along the outer conical surface 624 that establishes fluid flow communication between an external environment at the outer conical surface 624 and the lumen 650. When the suction adapter device 600 is coupled with the sheath 30 as shown in the arrangement of FIG. 8C, the fluid injection port 690 enables injection of fluid such as saline into the sheath 30 to lubricate the interior of the sheath 30 as the medical tube 20 is being drawn through the sheath 30. In the arrangement of FIG. 8C, the system can include a first suction adapter device 600A coupled to the second sheath portion 34 of the sheath 30, a second suction adapter device 600B coupled to the first sheath portion 32 of the sheath 30, and further includes a fluid source 90 that can include a nozzle 92 for insertion into the fluid injection port 690 of the second suction adapter device 600B. The fluid source 90 and associated nozzle 92 can inject a fluid 94 such as saline or another suitable liquid into the second sheath portion 34 of the sheath 30 to provide lubrication as the medical tube 20 is being drawn through the sheath 30 by the suction generation device 60 as shown. The first suction adapter device 600A, when arranged as shown in FIG. 8C engages with the suction tube 40 which can include the suction tube connector 50 such that the fluid injection port 690 is occluded by the suction tube connector 50 and/or the suction tube 40 so as not to let in air at the fluid injection port 690 of the first suction adapter device 600A. In some embodiments, as shown in FIG. 8B, the fluid injection port 690 includes a first port section 692A associated with the first face 612A of the first section 604A and a second port section 692B associated with the second face 612B of the second section 604B that, when coupled together, collectively define the fluid injection port 690. When drawing the medical tube 20 through the sheath 30 and when not injecting fluid into the fluid injection port 690 of the second suction adapter device 600B, the fluid injection port 690 can be manually occluded by a finger of a practitioner to maximize or otherwise modulate the suction force through the sheath 30.

The sheath connection portion 640 includes an outer surface 644 adjacent to the outer conical surface 624 of the nose portion 620. The outer surface 644 can be wide enough to provide a comfortable gripping area for a practitioner when handling the suction adapter device 600; in some embodiments, the first grip protrusion 686A of the first section 604A and the second grip protrusion 686B of the second section 604B can be present along the outer surface 644 of the sheath connection portion 640. The sheath connection portion 640 can further define a second terminal surface 646 having a protrusion 648 extending along the direction of elongation of the lumen 650. As shown, the protrusion 648 defines the second aperture 642 that communicates with the lumen 650, and can optionally include one or more barbs 649. In the embodiment of FIGS. 8A-8C, the protrusion 648 of the sheath connection portion 640 is configured for insertion into the first sheath portion 32 or the second sheath portion 34 of the sheath 30 (e.g., the first sheath aperture 33 of the first sheath portion 32 or the second sheath aperture 35 of the second sheath portion 34 of the sheath 30 can receive the protrusion 648 of the sheath connection portion 640) to establish fluid flow communication between the sheath lumen 36 of the sheath 30 and the lumen 650 of the suction adapter device 600. Further, the protrusion 648 can be secured within the sheath lumen 36 of the sheath 30 through friction-fit contact with the inner surface 38 of the sheath 30; in some embodiments, the one or more barbs 649 aid in maintaining this connection. The second terminal surface 646 provides an abutment surface for the terminal end of the first sheath portion 32 or the second sheath portion 34 of the sheath 30.

As shown in FIG. 8B, and with additional reference to the system 10 of FIGS. 2A-2G, the first aperture 622 of the nose portion 620 of the suction adapter device 600 defines a first diameter D1 and the second aperture 642 of the sheath connection portion 640 of the suction adapter device 600 defines a second diameter D2. As such, the lumen 650 can be of the first diameter D1 towards the first aperture 622 of the nose portion 620 and the second diameter D2 towards the second aperture 642 of the sheath connection portion 640. In the embodiment shown, the first diameter D1 can be significantly greater than the second diameter D2, as the first diameter D1 needs to accommodate the funnel 628 of the nose portion 620 and the second diameter D2 and the sheath connection portion 640 need to be small enough for insertion into the sheath lumen 36 of the sheath 30. As such, the lumen 650 can include a tapering portion 654 that transitions the diameter of the lumen 650 from the first diameter D1 to the second diameter D2.

During operation, after the medical tube 20 has been drawn through the sheath 30 and the first portion 22 of the medical tube 20 has arrived at the first sheath portion 32 of the sheath 30, the first portion 22 of the medical tube 20 remains within the lumen 650 of the suction adapter device 600 for lateral removal as discussed above with reference to FIGS. 2F and 2G (e.g., by separating the first section 604A from the second section 604B and accessing the lumen 650 therein).

In one aspect, the suction adapter device 100, 200, 300, 400, 500 or 600 shown in FIGS. 3A-8C can include a flexible rubber, silicon or plastic material. In a primary embodiment, the suction adapter device 100, 200, 300, 400, 500 or 600 can be reusable; as such, the suction adapter device 100, 200, 300, 400, 500 or 600 including associated surfaces and materials can be cleanable and robust in order to be re-used. In some embodiments, dimensions of the suction adapter device 100, 200, 300, 400, 500 or 600 can be configured for use across more than one specific application to accommodate variations in diameter of the sheath 30 or the suction tube 40 (FIGS. 1A-2G) that may arise between different applications (e.g., based on the type of procedure being applied and/or size of the body, as pediatric surgical procedures often use smaller devices than adult surgical procedures).

Further, the suction adapter device 100, 200, 300, 400, 500 or 600 shown in FIGS. 3A-8C and associated catheter placement system 10 shown in FIGS. 1A-2G and 8C can be applied for treatment of various diseases and/or conditions including but not limited to normal pressure hydrocephalus, obstructive hydrocephalus, pseudo tumor cerebri, congenital ventriculomegaly, chronic arachnoid cyst, and/or chronic and complex cerebrospinal fluid leak. In one aspect, the catheter placement system 10 can be used for treatment of hydrocephalus or another similar condition requiring a ventriculoperitoneal shunt (e.g., from a ventricle of the brain to the peritoneal cavity), where the first incision 2 is made near the brain and the second incision 4 is made at the abdomen as shown in FIGS. 1A-1C and where the medical tube 20 is a ventriculoperitoneal shunt catheter where the first portion 22 of the medical tube 20 is positioned at the peritoneal cavity of the body 1 and the second portion 24 of the medical tube 20 is positioned at a ventricle of the brain. With respect to other diseases and/or conditions mentioned above, the medical tube 20 can be a ventriculopleural shunt catheter, where the first portion 22 of the medical tube 20 is positioned at a pleural cavity of the body 1 and the second portion 24 is positioned at a ventricle of the brain. The medical tube 20 can be a ventriculoatrial shunt catheter, where the first portion 22 of the medical tube 20 is positioned at an atrium of a heart of the body 1 and the second portion 24 of the medical tube 20 is positioned at a ventricle of the brain. The medical tube 20 can be a cisternoperitoneal shunt catheter, where the first portion 22 of the medical tube 20 is positioned at a peritoneal cavity of the body 1 and the second portion 24 of the medical tube 20 is positioned at a subarachnoid cistern of the brain. The medical tube 20 can be a cisternopleural shunt catheter, where the first portion 22 of the medical tube 20 is positioned at the pleural cavity of the body 1 and the second portion 24 of the medical tube 20 is positioned at a subarachnoid cistern of the brain. The medical tube 20 can be a cisternoatrial shunt catheter, where the first portion 22 of the medical tube 20 is positioned at an atrium of a heart of the body 1 and the second portion 24 of the medical tube 20 is positioned at a subarachnoid cistern of the brain. In another aspect, the medical tube 20 can be employed and installed according to various embodiments and methods described herein for diversion of fluid from a cavity of the body at a first location of the body to an external environment at a second location of the body or for communicating fluid from an external environment at the first location to a cavity of the body at the second location.

### Methods

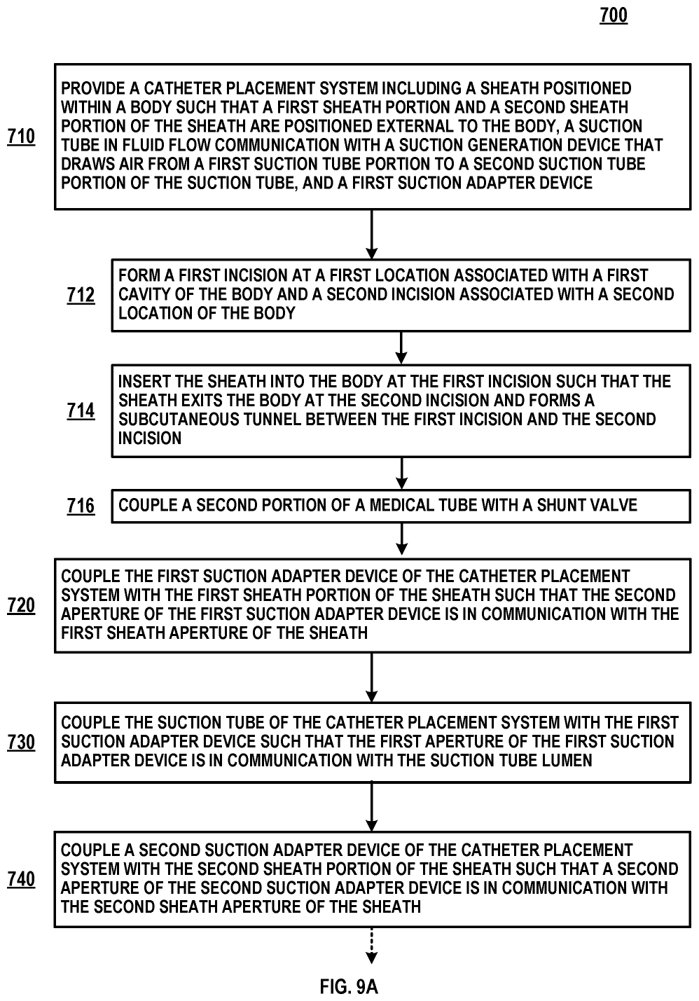

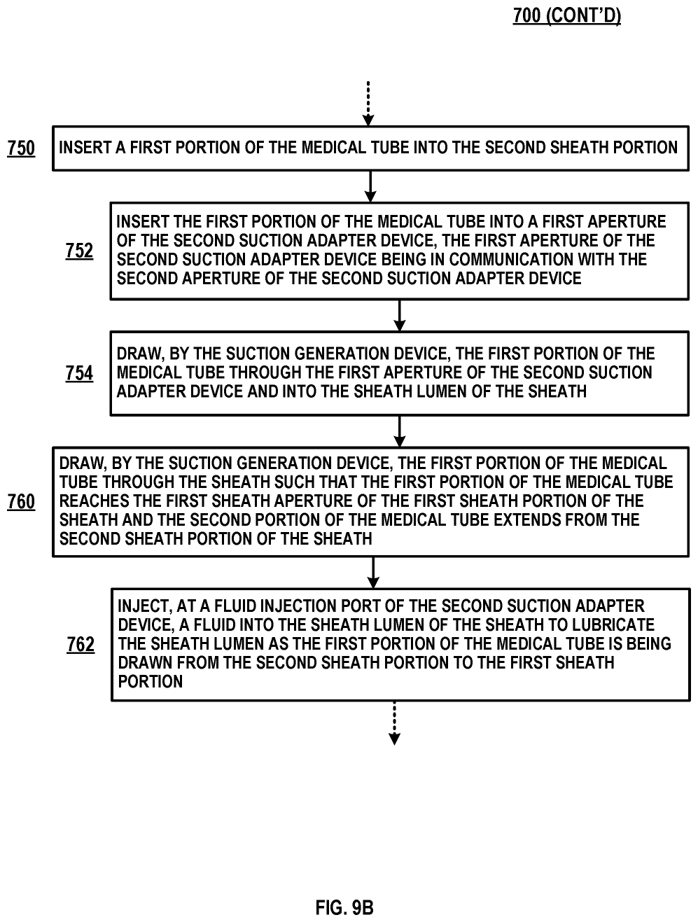

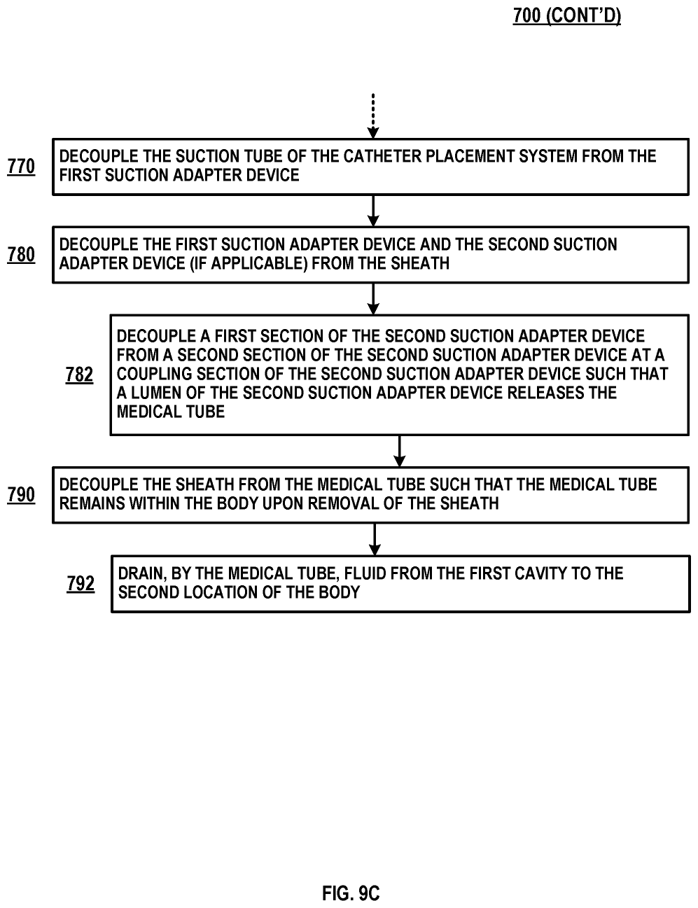

FIGS. 9A-9C illustrate a method 700 of drawing the medical tube 20 through the sheath 30 using the system 10 of FIGS. 1A-2G, and in particular, using the suction adapter device 100, 200, 300, 400, 500 or 600 of FIGS. 3A-8C.

Starting at FIG. 9A, block 710 of method 700 includes providing a catheter placement system including: a sheath positioned within a body such that a first sheath portion and a second sheath portion of the sheath are positioned external to the body, a suction tube in fluid flow communication with a suction generation device that draws air from a first suction tube portion to a second suction tube portion of the suction tube, and a first suction adapter device. Block 710 can include various sub-steps. Block 712 of method 700 can include forming a first incision at a first location associated with a first cavity of the body and a second incision associated with a second location of the body. In some embodiments, as discussed above, the first cavity can be a ventricle of the brain or a subarachnoid cistern of the brain; the second location can be a peritoneal cavity, a pleural cavity, and/or an atrium of the heart, and can alternatively be an external environment. As such, the catheter placement system and associated methods can be extended to treatment of other pathologies involving diversion of fluid from one cavity of the body to another cavity of the body or an external location of the body. Block 714 of method 700 can include inserting the sheath into the body at the first incision such that the sheath exits the body at the second incision and forms a subcutaneous tunnel between the first incision and the second incision. Block 716 of method 700 can include coupling a second portion of a medical tube with a shunt valve.

Block 720 of method 700 includes coupling the first suction adapter device of the catheter placement system with the first sheath portion of the sheath such that the second aperture of the first suction adapter device is in communication with the first sheath aperture of the sheath.

Block 730 of method 700 includes coupling the suction tube of the catheter placement system with the first suction adapter device such that the first aperture of the first suction adapter device is in communication with the suction tube lumen.

Block 740 of method 700 includes coupling a second suction adapter device of the catheter placement system with the second sheath portion of the sheath such that a second aperture of the second suction adapter device is in communication with the second sheath aperture of the sheath.

Continuing at FIG. 9B, block 750 of method 700 includes inserting a first portion of the medical tube into the second sheath portion, and can include one or more sub-blocks. Sub-block 752 of block 750 includes inserting the first portion of the medical tube into a first aperture of the second suction adapter device, the first aperture of the second suction adapter device being in communication with the second aperture of the second suction adapter device. Sub-block 754 of block 750 includes drawing, by the suction generation device, the first portion of the medical tube through the first aperture of the second suction adapter device and into the sheath lumen of the sheath.

Block 760 of method 700 includes drawing, by the suction generation device, the first portion of the medical tube through the sheath such that the first portion of the medical tube reaches the first sheath aperture of the first sheath portion of the sheath and a second portion of the medical tube extends from the second sheath portion of the sheath. Block 762 of method 700 includes injecting, at a fluid injection port of the second suction adapter device, a fluid into the sheath lumen of the sheath to lubricate the sheath lumen as the first portion of the medical tube is being drawn from the second sheath portion to the first sheath portion.

Continuing with FIG. 9C, block 770 of method 700 includes decoupling the suction tube of the catheter placement system from the first suction adapter device.

Block 780 of method 700 includes decoupling the first suction adapter device and the second suction adapter device (if applicable) from the sheath. Block 780 can include sub-block 782, which includes decoupling a first section of the second suction adapter device from a second section of the second suction adapter device at a coupling section of the second suction adapter device such that a lumen of the second suction adapter device releases the medical tube.

Finally, block 790 of method 700 includes decoupling the sheath from the medical tube such that the medical tube remains within the body upon removal of the sheath from the body. Block 792 of method 700 includes draining, by the medical tube, fluid from the first cavity to the second cavity.

As understood by one of skill in the art, the various embodiments encompassing suction connection systems described herein may be applied to treatment or surgical procedures for any number of diseases and/or conditions that require installation of medical tubes and the application is in no way limited to the aforementioned diseases and/or conditions.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A suction adapter device, comprising:
a body defining a first section and a second section, wherein the first section and the second section collectively define:
a lumen including a first lumen section defined by the first section and a second lumen section defined by the second section;
a coupling portion that removably couples the first section and the second section together;
a nose portion having a first aperture that communicates with the lumen; and
a sheath connection portion having a second aperture that communicates with the lumen.

2. The suction adapter device of claim 1, wherein the nose portion includes:
an outer conical surface configured to couple within a suction tube in communication with a suction generation device of a catheter placement system.

3. The suction adapter device of claim 1, wherein the nose portion includes:
a funnel that communicates with the first aperture of the nose portion.

4. The suction adapter device of claim 1, wherein the nose portion includes:
a fluid injection port that establishes fluid flow communication between an outer conical surface of the nose portion and the lumen.

5. The suction adapter device of claim 1, wherein the sheath connection portion includes:
a protrusion configured for insertion within a sheath of a catheter placement system.

6. The suction adapter device of claim 1, wherein the second aperture of the sheath connection portion is configured to receive a first sheath portion or a second sheath portion of a sheath of a catheter placement system.

7. The suction adapter device of claim 1, wherein the suction adapter device includes:
a hinge portion connecting the first section and the second section.

8. The suction adapter device of claim 1, wherein the suction adapter device includes:
a first grip protrusion associated with the first section; and
a second grip protrusion associated with the second section;
wherein manual actuation of the first grip protrusion away from the second grip protrusion separates the first section from the second section to expose the lumen therebetween.

9. The suction adapter device of claim 1, wherein the coupling portion includes:
a first face associated with the first section; and
a second face associated with the second section.

10. The suction adapter device of claim 9, wherein the first face and the second face include one or more coupling elements that removably couple the first face of the first section and the second face of the second section together.

11. The suction adapter device of claim 9, wherein the body includes a slit that separates the first face and the second face from one another and wherein the body includes a flexible material such that manual separation of the first face from the second face at the slit enables insertion or removal of a sheath of a catheter placement system from the lumen.

12. The suction adapter device of claim 1, the first aperture defining a first diameter and the second aperture defining a second diameter, wherein the lumen includes a tapering portion that transitions the lumen from the first diameter to the second diameter.

13. A catheter placement system, comprising:
a sheath defining a first sheath portion, a second sheath portion, and a sheath lumen, the sheath being configured for receipt of a medical tube at the second sheath portion;
a suction tube defining a first suction tube portion, a second suction tube portion, and a suction tube lumen, the second suction tube portion being in fluid flow communication with a suction generation device; and
a first suction adapter device including a lumen, a nose portion having a first aperture that communicates with the lumen, and a sheath connection portion having a second aperture that communicates with the lumen;
wherein the sheath connection portion of the first suction adapter device is configured for engagement with the first sheath portion of the sheath such that the lumen of the first suction adapter device is in fluid flow communication with the sheath lumen upon engagement of the sheath connection portion of the first suction adapter device with the first sheath portion of the sheath;
wherein the nose portion of the first suction adapter device is configured for engagement with the first suction tube portion of the suction tube such that the lumen of the first suction adapter device is in fluid flow communication with the suction tube lumen upon engagement of the nose portion of the first suction adapter device with the first suction tube portion of the suction tube; and
wherein the suction generation device generates a suction force that draws the medical tube from the second sheath portion to the first sheath portion when coupled with the suction tube, the first suction adapter device and the sheath.

14. The catheter placement system of claim 13, further comprising:
a second suction adapter device including a lumen, a nose portion having a first aperture that communicates with the lumen, and a sheath connection portion having a second aperture that communicates with the lumen;
wherein the sheath connection portion of the second suction adapter device is configured for engagement with the second sheath portion of the sheath.

15. The catheter placement system of claim 14, wherein the first aperture of the second suction adapter device includes a funnel that enables insertion of the medical tube into the lumen of the second suction adapter device.

16. The catheter placement system of claim 14, wherein the second suction adapter device comprises a body defining a first section and a second section removably coupled with one another, wherein the first section and the second section collectively define the lumen, the nose portion and the sheath connection portion of the second suction adapter device.

17. The catheter placement system of claim 14, wherein the second suction adapter device includes a fluid injection port that establishes fluid flow communication between an outer conical surface of the nose portion and the lumen such that injection of a fluid into the fluid injection port of the second suction adapter device lubricates the sheath lumen of the sheath as the medical tube is being drawn from the second sheath portion to the first sheath portion.

18. The catheter placement system of claim 13, wherein the second aperture of the sheath connection portion of the first suction adapter device is configured for engagement with the first sheath portion of the sheath by insertion of the first sheath portion of the sheath into the second aperture of the sheath connection portion of the first suction adapter device.

19. The catheter placement system of claim 13, wherein the sheath connection portion of the first suction adapter device includes a protrusion and wherein the sheath connection portion of the first suction adapter device is configured for engagement with the first sheath portion of the sheath by insertion of the protrusion of the first suction adapter device into the first sheath portion of the sheath.

20. The catheter placement system of claim 13, wherein the first suction tube portion of the suction tube includes a suction tube connector, the suction tube connector including a first connector aperture that communicates with the suction tube lumen of the suction tube, and wherein the nose portion of the first suction adapter device is configured for engagement with the first connector aperture of the suction tube connector in association with the suction tube.

21. A method, comprising:
providing a catheter placement system including:
a sheath defining a first sheath portion having a first sheath aperture, a second sheath portion having a second sheath aperture, and a sheath lumen in communication with the first sheath aperture and the second sheath aperture, the sheath being configured for receipt of a medical tube at the second sheath portion and the sheath being positioned within a body such that the first sheath portion and the second sheath portion are positioned external to the body;
a suction tube defining a first suction tube portion, a second suction tube portion, and a suction tube lumen, the second suction tube portion being in fluid flow communication with a suction generation device that draws air from the first suction tube portion to the second suction tube portion;
a first suction adapter device including a lumen, a nose portion having a first aperture that communicates with the lumen, and a sheath connection portion having a second aperture that communicates with the lumen;
coupling the first suction adapter device of the catheter placement system with the first sheath portion of the sheath such that the second aperture of the first suction adapter device is in communication with the first sheath aperture of the sheath;
coupling the suction tube of the catheter placement system with the first suction adapter device such that the first aperture of the first suction adapter device is in communication with the suction tube lumen;
inserting a first portion of the medical tube into the second sheath portion; and
drawing, by the suction generation device, the first portion of the medical tube through the sheath such that the first portion of the medical tube reaches the first sheath aperture of the first sheath portion of the sheath and a second portion of the medical tube extends from the second sheath portion of the sheath.

22. The method of claim 21, further comprising:
decoupling the suction tube of the catheter placement system from the first suction adapter device;
decoupling the first suction adapter device from the sheath; and
decoupling the sheath from the medical tube such that the medical tube remains within the body upon removal of the sheath from the body.

23. The method of claim 21, further comprising:
coupling a second suction adapter device of the catheter placement system with the second sheath portion of the sheath such that a second aperture of the second suction adapter device is in communication with the second sheath aperture of the sheath; and
inserting the first portion of the medical tube into a first aperture of the second suction adapter device, the first aperture of the second suction adapter device being in communication with the second aperture of the second suction adapter device.

24. The method of claim 23, further comprising:
drawing, by the suction generation device, the first portion of the medical tube through the first aperture of the second suction adapter device and into the sheath lumen of the sheath.

25. The method of claim 23, further comprising:
decoupling a first section of the second suction adapter device from a second section of the second suction adapter device at a coupling section of the second suction adapter device such that a lumen of the second suction adapter device releases the medical tube.

26. A method of treatment of a disease or condition using a catheter placement system, comprising:
providing a catheter placement system including a sheath having a first sheath portion and a second sheath portion, a first suction adapter device, a suction tube, a suction generation device, and a medical tube having a first portion and a second portion;
positioning the medical tube within a body by:
forming, by the sheath, a subcutaneous tunnel between a first incision associated with a first cavity of the body and a second incision associated with a second location of the body;
coupling the first suction adapter device to the first sheath portion and the suction tube of the catheter placement system in fluid flow communication with the suction generation device for establishment of fluid flow communication between the suction generation device and the first sheath portion of the sheath; and
drawing, by the suction generation device, the first portion of the medical tube from the second sheath portion to the first sheath portion such that the first portion of the medical tube is positioned at the first sheath portion of the sheath and such that the second portion of the medical tube is positioned at the second sheath portion of the sheath; and
draining, by the medical tube, fluid from the first cavity to the first portion of the medical tube.

27. The method of claim 26, further comprising:
coupling a second suction adapter device of the catheter placement system to the second sheath portion prior to insertion of the first portion of the medical tube into the second sheath portion of the sheath.

28. The method of claim 26, further comprising:
securing the first portion of the medical tube within the body at a second cavity of the body associated with the second location to establish fluid flow communication between the first cavity and the second cavity.

29. The method of claim 26, further comprising:
removing the sheath from the subcutaneous tunnel at the second incision such that the medical tube remains within the subcutaneous tunnel.

30. The method of claim 26, wherein the medical tube is a ventriculoperitoneal shunt catheter and wherein the first cavity is a ventricle of a brain of the body and wherein the second location of the body is a peritoneal cavity of the body.

31. The method of claim 26, wherein the medical tube is a ventriculopleural shunt catheter and wherein the first cavity is a ventricle of a brain of the body and wherein the second location of the body is a pleural cavity of the body.

32. The method of claim 26, wherein the medical tube is a ventriculoatrial shunt catheter and wherein the first cavity is a ventricle of a brain of the body and wherein the second location of the body is an atrium of a heart of the body.

33. The method of claim 26, wherein the medical tube is a cisternoperitoneal shunt catheter and wherein the first cavity is a subarachnoid cistern of a brain of the body wherein the second location of the body is a peritoneal cavity of the body.

34. The method of claim 26, wherein the medical tube is a cisternopleural shunt catheter and wherein the first cavity is a subarachnoid cistern of a brain of the body and wherein the second location of the body is a pleural cavity of the body.

35. The method of claim 26, wherein the medical tube is a cisternoatrial shunt catheter and wherein the first cavity is a subarachnoid cistern of a brain of the body and wherein the second location of the body is an atrium of a heart of the body.

36. The method of claim 26, wherein the second portion of the medical tube is positioned external to the body at the second location.
